SEQUENCE LISTING (10 sequences)

SEQ ID NO: 1 - protein, 11 aa, human
  MISC_FEATURE (1)..(11): Corresponds to residues 225-235 in the full-length wt-APC protein
EYDLRRWEKWE

SEQ ID NO: 2 - protein, 11 aa, human
  MISC_FEATURE (5)..(6): Residues 5-6 are R-to-A mutations corresponding to residues 229-230 in the full-length wt-APC protein
EYDLAAWEKWE

SEQ ID NO: 3 - protein, 6 aa, human
  MISC_FEATURE (1)..(6): Corresponds to residues 189-194 in the full-length wt-APC protein
DSKKKL

SEQ ID NO: 4 - protein, 6 aa, human
  MISC_FEATURE (3)..(5): Residues 3-5 are K-to-A mutations corresponding to residues 191-193 in the full-length wt-APC protein
DSAAAL

SEQ ID NO: 5 - protein, 6 aa, human
  MISC_FEATURE (1)..(6): Corresponds to residues 227-232 in the full-length wt-APC protein
DLRRWE

SEQ ID NO: 6 - protein, 6 aa, human
  MISC_FEATURE (3)..(4): Residues 3-4 are R-to-A mutations corresponding to residues 229-230 in the full-length wt-APC protein
DLAAWE

SEQ ID NO: 7 - protein, 7 aa, human
  MISC_FEATURE (1)..(7): Corresponds to residues 189-195 in the full-length wt-APC protein
DSKKKLA

SEQ ID NO: 8 - protein, 7 aa, human
  MISC_FEATURE (3)..(5): Residues 3-5 are K-to-A mutations corresponding to residues 191-193 in the full-length wt-APC protein
DSAAALA

SEQ ID NO: 9 - protein, 5 aa, human
  MISC_FEATURE (3)..(3): Residue 3 is a S-to-A mutation corresponding to position 360 in the full-length wt-APC protein
GDAGG

SEQ ID NO: 10 - protein, 419 aa, Human
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

(19) United States
(12) Patent Application Publication
(10) Pub. No.: US 2010/0284997 A1
(43) Pub. Date: Nov. 11, 2010

(54) DOSING REGIMEN OF ACTIVATED PROTEIN C AND VARIANTS HAVING REDUCED ANTICOAGULANT ACTIVITY

(76) Inventors: John H. Griffin, Del Mar, CA (US); Hartmut Weiler, Delafield, WI (US)

Correspondence Address: LOEB & LOEB LLP, 321 N. CLARK STREET, SUITE 2300, CHICAGO, IL, 60654, US

(21) Appl. No.: 12/447,845

(22) PCT Filed: Oct. 31, 2007
(86) PCT No.: PCT/US07/83249

§ 371 Date: Jul. 7, 2010

Related U.S. Application Data
(60) Provisional application No. 60/863,725, filed on Oct. 31, 2006.

(51) Int. Cl. A61K 38/48; C12N 9/48; A61P 31/04; A61P 7/02

(52) U.S. Cl. 424/94.64; 435/212

(57) ABSTRACT

Recombinant activated protein C (APC) and APC variants with reduced anticoagulant activity were used to reduce mortality in murine models of sepsis. These models included endotoxemia and bacteremia models. We discovered that single or multiple bolus doses of APC, especially of APC variants such as RR230/231AA-APC, KKK192-194AAA-APC and 5A-APC (containing the combination of mutations present in the first two APC variants) given as a single bolus reduces 7-day mortality of mice given lethal doses of endotoxin. Administrations of a single bolus of 5A-APC after the initiation of sepsis also reduces mortality caused by LPS. 5A-APC with ≦8% of normal anticoagulant activity (which has reduced risk of bleeding) reduces mortality when given as two bolus administrations at 3 hours and then at 10 hours after initiation of bacterial infection, i.e. after onset of sepsis. This shows, first, that one or more bolus injections of APC or of APC variants, especially 5A-APC, can reduce mortality when given beginning hours after the onset of sepsis and, second, that it is not necessary to administer APC as a continuous infusion which is the current standard of practice because one or more bolus administrations can reduce mortality. Furthermore, dosages of approximately 0.06 to 0.4 mg/kg of APC and APC variants are identified to be sufficient to reduce mortality in sepsis.

This invention was made with the U.S. Government support under Contract Nos. HL31950, HL52246, and HL60655 by the National Institutes of Health. The U.S. Government has certain rights to this invention.

### FIELD OF THE INVENTION

The present invention relates to dosing regimens of wild type and variants (mutants) of recombinant protein C and activated protein C, an enzyme that normally has anti-thrombotic, anti-inflammatory, and anti-apoptotic activities. The recombinant activated protein C mutants of the invention have markedly reduced anticoagulant activity but retain near normal anti-apoptotic (cytoprotective) activity. The invention relates to administering to a subject a dose of APC mutants with reduced anticoagulant activity but normal or near normal cytoprotective activities as a single bolus or brief infusion or as two boluses or brief infusions. More specifically, the invention relates to methods of treating sepsis by administering to a subject APC mutants with reduced anticoagulant activity but normal or near normal cytoprotective activities as a single bolus or brief infusion or as two boluses or brief infusions.

### BACKGROUND OF THE INVENTION

Protein C is a member of the class of vitamin K-dependent serine protease coagulation factors. Protein C was originally identified for its anticoagulant and profibrinolytic activities. Protein C circulating in the blood is an inactive zymogen that requires proteolytic activation to regulate blood coagulation through a complex natural feedback mechanism. Human protein C is primarily made in the liver as a single polypeptide of 461 amino acids. This precursor molecule is then post-translationally modified by (i) cleavage of a 42 amino acid signal sequence, (ii) proteolytic removal from the one-chain zymogen of the lysine residue at position 155 and the arginine residue at position 156 to produce the two-chain form (i.e., light chain of 155 amino acid residues attached by disulfide linkage to the serine protease-containing heavy chain of 262 amino acid residues), (iii) carboxylation of the glutamic acid residues clustered in the first 42 amino acids of the light chain resulting in nine gamma-carboxyglutamic acid (Gla) residues, and (iv) glycosylation at four sites (one in the light chain and three in the heavy chain). The heavy chain contains the serine protease triad of Asp257, His211 and Ser360.

Recombinant forms of protein C can be produced with a selected chemical structure (e.g., native, mutant, or polymorphic). As an illustration, a gene encoding human protein C is described in U.S. Pat. No. 4,775,624 and can be used to produce recombinant human protein C as described in U.S. Pat. No. 4,981,952. Human protein C can be recombinantly produced in tissue culture and activated as described in U.S. Pat. No. 6,037,322. Natural human protein C can be purified from plasma, activated, and assayed as described in U.S. Pat. No. 5,084,274. The nucleotide and amino acid sequence disclosed in these patents may be used as a reference for protein C.

Similar to most other zymogens of extracellular proteases and the coagulation factors, protein C has a core structure of the chymotrypsin family, having insertions and an N-terminus extension that enable regulation of the zymogen and the enzyme. Of interest are two domains with amino acid sequences similar to epidermal growth factor (EGF). At least a portion of the nucleotide and amino acid sequences for protein C from human, monkey, mouse, rat, hamster, rabbit, dog, cat, goat, pig, horse, and cow are known, as well as mutations and polymorphisms of human protein C (see GenBank accession P04070). Other variants of human protein C are known which affect different biological activities. Notably, murine protein C contains a single amino acid insertion at residue 160 compared with the human protein C.

Activation of protein C is mediated by thrombin. Thrombin binds to thrombomodulin, a membrane-bound thrombin receptor on the luminal surface of endothelial cells, thereby blocking the procoagulant activity of thrombin via its exosite I, and enhancing its anticoagulant properties, i.e., activating protein C. As an anticoagulant, activated protein C (APC), aided by its cofactor protein S, cleaves the activated cofactors factor Va and factor VIIIa, which are required in the intrinsic coagulation pathway to sustain thrombin formation (Esmon et al., Biochim. Biophys. Acta., 1477:349-360, 2000a), to yield the inactivated cofactors factor Vi and factor VIIIi.

The thrombin/thrombomodulin complex mediated activation of protein C is facilitated when protein C binds to the endothelial protein C receptor (EPCR), which localizes protein C to the endothelial cell membrane surface. When complexed with EPCR, APC's anticoagulant activity is inhibited; APC expresses its anticoagulant activity when it dissociates from EPCR, especially when bound to negatively charged phospholipids on activated platelet or endothelial cell membranes.

Components of the protein C pathway contribute not only to anticoagulant activity, but also to anti-inflammatory functions (Griffin et al., Sem. Hematology, 39:197-205, 2002). The anti-inflammatory effects of thrombomodulin, recently attributed to its lectin-like domain, can protect mice against neutrophil-mediated tissue damage (Conway et al., J. Exp. Med. 196:565-577, 2002). APC provides EPCR-dependent protection against the lethal effects of E. coli infusion in baboons (Taylor et al., Blood, 95:1680-1686, 2000) and can downregulate proinflammatory cytokine production and favorably alter tissue factor expression or blood pressure in various models (Shu et al., FEBS Lett. 477:208-212, 2000; Isobe et al., Circulation, 104:1171-1175, 2001; Esmon, Ann. Med., 34:598-605, 2002).

Inflammation is the body's reaction to injury and infection. Three major events are involved in inflammation: (1) increased blood supply to the injured or infected area; (2) increased capillary permeability enabled by retraction of endothelial cells; and (3) migration of leukocytes out of the capillaries and into the surrounding tissue (hereinafter referred to as cellular infiltration) (Roitt et al., Immunology, Grower Medical Publishing, New York, 1989).

APC has not only anticoagulant and anti-inflammatory activities but also anti-apoptotic activity. EPCR has been found to be a required cofactor for the anti-apoptotic activity of APC in certain cells, as APC activation of protease activated receptor-1 (PAR-1) is EPCR-dependent (Riewald et al., Science, 2296:1880-1882, 2002; Cheng et al., Nat. Med., 9:338-342, 2003; Mosnier and Griffin, Biochem. J., 373:65-70, 2003). APC anticoagulant activity involves inactivation of factors Va and VIIIa whereas cytoprotection by APC involves two receptors, EPCR and PAR-1. Significant levels of EPCR have been found, for example, in hematopoietic stem cells in bone (Balazs, Blood 107:2317-21, 2006).

As used herein, the term sepsis is defined as a suspected or proven infection plus a systemic inflammatory response syndrome (for example, fever, tachycardia, tachypnea, or leukocytosis); as used herein, the phrase severe sepsis is defined as sepsis with organ dysfunction (for example, hypotension, hypoxemia, oliguria, metabolic acidosis, thrombocytopenia, or obtundation) (Russell, New Engl. J. Med. 355(16):1699-713, 2006). Importantly, in many cases of sepsis, it is not possible to establish the cause of an infection.

Numerous in vivo studies document the beneficial effects of APC. Recombinant activated protein C (rAPC), similar to Xigris (Eli Lilly & Co.), is approved for treating severe sepsis (Hotchkiss and Karl, NEJM, 348: 138-150, 2003; Russell, New Engl. J. Med. 355(16):1699-713, 2006) and it may eventually have other beneficial applications. However, clinical studies have shown APC treatment to be associated with increased risk of serious bleeding. This increased risk of bleeding presents a major limitation of APC therapy. If APC's beneficial effects in sepsis can be attributed to its anti-inflammatory and cell survival activities, a compound that retains the beneficial anti-apoptotic or cytoprotective activity but has a less anticoagulant activity is desirable. Moreover, the full anticoagulant activity of APC might impair mechanisms involving the beneficial fibrin-dependent clearance of bacteria.

Current treatment methods employing recombinant activated protein C involve continuous infusions. It is an object of this invention to improve and simplify the use of APC or APC variants for treatment of sepsis.

### SUMMARY OF THE INVENTION

Accordingly, it is an object of this invention to provide a novel dosing regimen of recombinant APC as a therapeutic for use in alleviating or preventing cell damage associated at least in part with apoptosis. More specifically, it is an object of this invention to provide a novel dosing regimen of variants (mutants) of recombinant APC as therapeutics for use in alleviating or preventing cell damage associated at least in part with apoptosis. The invention is directed to dosing regimens of variants of recombinant APC that provide reduced anticoagulant activity relative to anti-apoptotic activity compared to wild-type. More specifically, the invention is directed to bolus administration of APC or APC variants for treatment of sepsis.

Examples of useful recombinant murine APC mutants for use in the novel dosing regimen are KKK192-194AAA-APC (mutation of lysines 192, 193 and 194 to alanines (murine “3K3A-APC”)), RR230/231AA-APC (mutation of arginines 230 and 231 to alanines), and RR230/231AA plus KKK192-194AAA-APC (combination of mutations of arginines 230 and 231 to alanines and lysines 192, 193 and 194 to alanines (hereinafter murine “5A-APC”)). Corresponding useful recombinant human APC mutants for use in the novel dosing regimen are KKK191-193AAA-APC (mutation of lysines 191, 192 and 193 to alanines (human “3K3A-APC”)), RR229/230AA-APC (mutation of arginines 229 and 230 to alanines), and RR229/230AA plus KKK191-193AAA-APC (combination of mutations of arginines 229 and 230 to alanines and lysines 191, 192 and 193 to alanines (hereinafter human “5A-APC”)).

These exemplary APC variants retain the desirable property of normal anti-apoptotic, cytoprotective activity but provide significantly reduced risk of bleeding or serious bleeding, given their reduced anticoagulant activity.

Activated protein C (APC) reduces mortality in severe sepsis patients. (Bernard et al., New Engl. J. Med. 344:699-709, 2001a). APC exerts anticoagulant activities and acts directly on cells with multiple cytoprotective effects. (see, e.g., Cheng et al. Nat. Med., 9:338-42, 2003; Domotor et al., Blood, 101:4797-4801, 2003; Mosnier and Griffin, Biochem. J., 373:65-70, 2003). To clarify mechanisms for APC's reduction of mortality, we studied two murine sepsis models and used mice deficient in known APC receptors and recombinant APC variants with attenuated anticoagulant but normal cytoprotective activities. (Mosnier et al., Blood. 104:1740-1745, 2004).

We discovered that murine wild type APC rescues mice from lipopolysaccharide (LPS)-induced death and that two receptors, endothelial cell protein C receptor (EPCR) (Fukudome and Esmon, J. Biol. Chem., 270:5571-5577, 1995) and protease activated receptor-1 (PAR-1) (Coughlin, Nature 407, 258-264, 2000; Mosnier et al., Blood. 104:1740-1745, 2004), are essential for these survival benefits. APC variants such as RR230/231AA-APC, KKK192-194AAA-APC, and 5A-APC with reduced anticoagulant but normal cytoprotective activity effectively reduce LPS-induced mortality.

Furthermore, a single dose of APC reduced kidney damage and liver damage caused by LPS. Thus, APC is useful for preventing or reducing damage to kidney or to liver in the setting of sepsis. For peritoneal Staphylococcus aureus-induced mortality, wild type APC actually increased mortality when given at the time of initiation of infection. When administered after the onset of bacterial sepsis, the APC variant, 5A-APC, with greatly attenuated anticoagulant activity prevented mortality when administered as two boluses at two times at hours after the onset of sepsis. Thus, we discovered that APC's cytoprotective activity is critical for mortality reduction while APC's full anticoagulant activity is not required.

APC administered as a single dose prevents liver damage and kidney damage. APC variants with reduced anticoagulant activity but normal or near normal cytoprotective activities, i.e., anti-inflammatory, anti-apoptotic, anti-adhesive and/or endothelial barrier stabilization activities, are useful to reduce mortality in sepsis when administered as a single bolus or brief infusion or as two boluses or brief infusions.

This invention improves and simplifies the use of APC or APC variants for various treatments, particularly treatment of sepsis, because lengthy and continuous infusions are avoided. Instead, only one or more bolus administrations, or a single brief infusion or brief infusions, beginning hours after the onset of sepsis are needed to reduce death caused by sepsis. This allows further dosing regimens involving multiple, discrete injections, rather than continuous infusions, of APC or APC variants to be used for treating sepsis.

While application of APC or APC variants in accordance with this invention is particularly effective in the treatment of sepsis and severe sepsis, it should be recognized that application of APC or APC variants in accordance with this invention may also be used in the treatment of endotoxemia and bacteremia.

This invention may be used in the treatment of bacterial infections including, by way of example, necrotizing fascitis. Treatment of other bacterial infections in accordance with this invention may also include Staphylococcus aureus and Group A streptococcus. Application of APC or APC variants in accordance with this invention may also be useful in aiding in wound healing and repair by promoting endothelial cell migration and proliferation. Beneficial effects of APC in regenerative processes, e.g., angiogenesis and wound healing, have been demonstrated. APC can induce endothelial cell proliferation and angiogenesis in vitro and in vivo, which was dependent on EPCR and PAR-1. (Uchiba M, Okajima K, Oike Y et al. Circ. Res. 95:34-41, 2004) APC can stimulate keratinocyte proliferation and migration and APC can contribute to extracellular matrix degradation by activation of the gelatinase, matrix metalloproteinase-2 (MMP-2). (Xue M, Thompson P, Kelso I, Jackson C. Exp Cell Res. 299:119-127, 2004; Nguyen N, Arkell J, Jackson C J. J Biol. Chem. 275:9095-9098, 2000; Xue M, Thompson P, Sambrook P N, March L, Jackson C J. Clin Hemorheol Microcirc. 34:153-61, 2006; Xue M, Campbell D, Sambrook P N, Fukudome K, Jackson C J. J Invest Dermatol. 125:1279-85, 2005.)

Application of APC or APC variants in accordance with this invention may be used to treat ailments in conjunction with, or in the place of, other prescribed medical treatments including treatments in conjunction with antibiotics.

APC and APC variants utilized in accordance with this invention may be of the human or murine varieties, or other varieties. APC or APC variants in accordance with this invention may be applied to a subject of any species of mammal, more particularly human, monkey, baboon, mouse, rat, hamster, rabbit, dog, cat, goat, sheep, pig, horse, or cow, even more particularly human, monkey, baboon, even more particularly human. APC or APC variants in accordance with this invention may be applied to a subject, to cells from a subject, or to an organ from a subject.

In one embodiment of the invention, a dose of activated protein C or a variant of activated protein C is provided to a subject wherein said dose is given as one bolus administration or in repeated bolus administrations. In another embodiment of the invention, a dose of a prodrug form of activated protein C or a prodrug form of a variant of activated protein C is provided to a subject wherein said dose is given as one bolus administration or in repeated bolus administrations.

In one embodiment, a dose of activated protein C or a variant of activated protein C is provided to a subject wherein said dose is given as one bolus administration. In one embodiment, a dose of activated protein C or a variant of activated protein C is provided to a subject wherein said dose is administered by bolus over the course of about 20 minutes. In another embodiment, a dose of activated protein C or a variant of activated protein C is provided to a subject wherein said dose is given twice in two bolus administrations. In yet another embodiment, a dose of activated protein C or a variant of activated protein C is provided to a subject wherein said dose is given as a brief infusion. In yet another embodiment, a dose of activated protein C or a variant of activated protein C is provided to a subject in each of several brief infusions.

In a preferred embodiment, the variant of activated protein C is selected from the group consisting of human 5A-APC or murine 5A-APC. In a preferred embodiment, the dose of activated protein C or a variant of activated protein C is about 0.06 mg to about 0.4 mg per kilogram of a subject's body mass, preferably, the dose is about 0.067 mg to about 0.33 mg per kilogram of a subject's body mass, more preferably, the dose is about 0.08 to about 0.33 mg per kilogram of a subject's body mass. It will be understood by one of ordinary skill in the art that the dose may be tailored to meet the needs of an individual subject based on various physiological principles, such as delivering an amount of activated protein C variant that achieves the desired cytoprotective effect, but with reduced risk for bleeding due to reduced anticoagulant activity. Accordingly, it should be recognized that doses may vary depending on the species of the subject or disease to be treated, e.g., the dose suitable for a human or other subject may vary by a factor of 2 or more from doses suitable for a mouse.

In another preferred embodiment, the variant of activated protein C has anticoagulant activity and cytoprotective activity, said activated protein C further having a protease domain comprising surface loops; wherein said activated protein C includes at least one mutation that differentially affects the activated protein C's anticoagulant activity and cytoprotective activity, said at least one mutation being in at least one amino acid residue of a surface loop of said protease domain; wherein the said surface loop is selected from the group consisting of loop 37 and calcium loop; and wherein said at least one mutation results in the anticoagulant activity, but not the cytoprotective activity, being reduced relative to a wild-type recombinant activated protein C.

In a preferred embodiment, the dose is provided to a subject that has a bacterial infection. In another preferred embodiment, the subject has endotoxemia.

Given the risk of bleeding associated with wild type activated protein C therapy, the APC mutants of this invention offer advantages over currently available wild-type recombinant APC. Therefore, APC mutants of the invention are expected to provide superior therapy, either alone or adjunctive to other agents, whenever APC might be used for its anti-inflammatory or anti-apoptotic or cell survival activities, rather than purely for its anticoagulant activity.

### DESCRIPTION OF DRAWINGS

FIG. 1: Recombinant Murine Activated Protein C protects mice from lethal effects of endotoxemia. A. Mice (n=20 per group except for the S360A-APC group where n=10) received either 10 μg of APC (), 2 μg of APC (▴), 10 μg of active site mutant S360A-APC (□) or PBS (◯) for a 20 minute period prior to receiving a LD50 dose of LPS via intraperitoneal injection. B. Mice (n=10 per group) received either 10 μg of APC (), or PBS (◯) for a 20 minute period prior to receiving an LD90 dose of LPS. Mice were monitored over a 7 day period for survival. Statistical significance was measured using a log-rank test.

FIG. 2: No significant histological differences seen between APC treated/LPS challenged mice and LPS challenged mice. Hematoxylin and eosin stained sections of spleen 400×, Liver 400× and Lung 400× from control mice A, D, and G; LPS challenged mice for 24 hrs B, E, and H; and APC treated/LPS challenged mice C, F, and I.

FIG. 3: Significant differences in the amount of apoptosis present in LPS challenged mice compared to APC infused/LPS challenged mice after 24 hours. TUNEL assay was performed on liver sections (200×) taken from control mice A (Hoechst) and D (TUNEL); LPS challenged mice B and E; and APC infused/LPS challenged mice C and F. G. Measurement of apoptosis in liver (solid bars), lung (hatched bars), and spleen tissue (open bars) from counting apoptotic bodies from numerous tissue sections. H. Measurement of Caspase-3 activity based on a fold increase from wild-type controls. (* p≦0.05 [Student t test]).

FIG. 4: Dextran infused kidney tissue samples indicate a significant difference in vascular permeability after 8 hours of LPS challenge compared to control and APC treated/LPS challenged mice. Mice were infused with high-molecular weight (FITC labeled) dextran and low-molecular weight (rhodamine labeled) dextran 8 hrs after LPS challenge. A-C. Kidney tissue viewed under 200× from various mice. The thickness of the rhodamine (red) band indicates vascular permeability. D. Measurement of vascular permeability based on a ratio of rhodamine thickness to vessel diameter in kidney tissue. Statistical significance was measured using a student t test.

FIG. 5: Analysis of EPCR^{δ/δ} mice and PAR1^{-/-} mice treated with APC in the presence of endotoxin. A. Survival analysis of EPCR^{δ/δ} mice (n=12 per group) treated with either 10 μg of APC () or PBS (◯) followed by an i.p. injection of 40 mg/kg of LPS. B. Survival analysis of PAR1^{-/-} mice (n=17 per group) treated with either 10 μg of APC () or PBS (◯) followed by an i.p. injection of 40 mg/kg of LPS. Statistical significance was measured using a log-rank test. Mice were monitored 7 days for survival.

FIG. 6: Thrombin-Antithrombin complex levels were determined to measure the level of thrombin generation in mice given endotoxin and treated with various forms of APC. A. Mice (n=5 per group) were infused for 20 min with the various forms of APC at 10 μg () and 2 μg (◯) doses or PBS (▴) and given LPS via i.p. injection 5 minutes after initiation of APC infusion. Blood was removed from mice 2 hours thereafter for measurement of TAT levels. B. Mice (n=5 per group) were given LPS via i.p. injection and rested for 2 hours. Mice were then treated with various forms of APC at 10 μg () and 2 μg (◯) doses or PBS (▴), and then rested for an additional 30 minutes until blood was collected for measurement of TAT levels. (p values in the top of each panel are for TAT level comparisons for various APC low doses (2 μg) compared to LPS alone; NS indicates Not Significant when low dose APC (2 μg) effects were compared to LPS alone effects [Student t test]. p values for “H vs. L” in the bottom of each panel indicate comparisons of the effect on TAT levels of higher dose APC (10 μg) to the effect of lower dose APC (2 μg).

FIG. 7: Murine APC and APC variants protect mice from lethal effects of endotoxemia. A. Mice (n=10 per group) were given a bolus i.v. injection of either 10 μg (, solid line) or 2 μg (◯) dose of RR230/231AA-APC (double point mutant) or no APC (PBS buffer alone) (□) followed by an i.p. injection of 40 mg/kg dose of LPS. B. Mice (n=10 per group) were given either 10 μg (, solid line) or 2 μg (◯) of 5A-APC (5-point mutant APC) or no APC (PBS buffer alone) (□) which was administered over 20 minutes followed by an i.p. injection of 40 mg/kg dose of LPS. C. Mice (n=10 per group were given a i.v. bolus of either 2 μg of 5A-APC (5-point mutant APC) () or control buffer alone (PBS) (□) which was administered at 3 hours (indicated by arrow) after receiving a 40 mg/kg dose of LPS via intraperitoneal injection. Mice were monitored over a 7 day period for survival. Statistical significance was measured using a log-rank test.

FIG. 8: Effects of various APC preparations on mortality of mice given an LD50 dose of S. aureus. A. Survival analysis of mice (n=10 per group) given an i.v. bolus dose of either 10 μg of APC (◯), RR230/231AA-APC (□), 5A-APC (Δ) or buffer alone (PBS) () and given 1×10⁸ S. aureus via an i.p. injection. B. Survival analysis of mice (n=10 per group) given an i.v. bolus dose of either 2 μg of wild type APC (◯), RR230/231AA-APC (□), 5A-APC (Δ) or buffer alone (PBS) () and given 1×10⁸ S. aureus via an i.p. injection. C. Survival analysis of mice (n=20 per group) given an i.v. bolus dose of either 2 μg of APC (◯), 5A-APC (Δ) or buffer alone (PBS) () at 0 hours and 10 hours post-infection (indicated by arrows) and given 1×10⁸ S. aureus via an i.p. injection at 0 hours. D. Survival analysis of mice (n=10 per group) given an i.v. bolus dose of either 2 μg of 5A-APC (Δ) or buffer alone (PBS) () at 3 hours and 10 hours post-infection (indicated by arrows) and given 1×10⁸ S. aureus via an i.p. injection at 0 hours. Mice were monitored over 7 days for survival. Statistical significance was measured using a log-rank test.

FIG. 9: The anticoagulant activity of murine APC variants compared to that of wild type (wt) murine APC was determined using activated partial thromboplastin time (APTT) clotting assays. The APTT protocol involved mixing the following reagents and determining the clotting times: 25 μl human plasma (from George King (GK) company); 25 μl murine wt APC (designated mAPCwt in figure) or the APC variants 230/231-APC (mAPC2m in figure), 3K3A-APC (mAPC3m in figure), or 5A-APC (mAPC5m in figure); 25 μl Platelin LS reagent; followed by 3 min incubation at 37 degrees C.; followed by addition of 50 μl 30 mM CaCl₂. The clotting time was recorded as time to clot formation after addition of CaCl₂. The mouse APC variants all had markedly reduced anticoagulant activity compared to wild type APC, with the 5A-APC having the lowest activity. The 5A-APC had no detectable activity at concentrations below 6 μg/ml, but at 12 μg/ml it was detectably active though significantly lower than the other APC mutants. Based on the initial slope of wild type APC and the observed values for APC mutants at 12.5 μg/ml, it is estimated that the anticoagulant activity of 230/231-APC, 3K3A-APC, and 5A-APC is 24%, 15% and 8%, respectively, of wt APC.

FIGS. 10-12: Various measurements from the various treatments in EPCR^{∂/∂} mice and PAR1^{-/-} mice.

### DETAILED DESCRIPTION OF THE INVENTION

Activated protein C (APC) has traditionally been regarded as an anticoagulant enzyme in the coagulation cascade, inhibiting thrombin formation and subsequent fibrin-clot formation by inactivating the cofactors factor Va and factor VIIIa (Esmon, supra, 2000a). However, APC also has the remarkable ability to reduce mortality in severe sepsis (Bernard et al., supra, 2001a; Bernard et al., Crit. Care Med., 29:2051-59, 2001b; Hinds, Brit. Med. J., 323:881-82, 2001; Kanji et al., Pharmacother., 21:1389-1402, 2001), while other anticoagulants such as antithrombin III and tissue factor pathway inhibitor have failed in this capacity (Warren et al., supra, 2001; Abraham et al., supra, 2001). This property of APC has peaked investigators' interest in the less extensively studied direct anti-inflammatory and anti-apoptotic activities attributed to APC (see, e.g., Cheng et al. Nat. Med., 9:338-42, 2003; Domotor et al., Blood, 101:4797-4801, 2003; Fernandez et al., Blood Cells Mol. Dis., 30:271-276, 2003; Esmon, J. Autoimmun., 15:113-116, 2000b). APC also has potential to protect the brain from damage caused by ischemic stroke (Cheng et al., supra, 2003; Esmon Thrombos Haemostas, 83:639-643, 2000c).

A major concern for the use of APC as a therapeutic is an increased risk of bleeding complications (Bernard et al., supra, 2001a, Bernard et al., supra, 2001b) due to APC anticoagulant activity. The APC variants of this invention solve this problem by having reduced anticoagulant activity over endogenous APC or wild-type recombinant APC, while retaining beneficial anti-apoptotic activity. APC variants having these useful characteristics are described in Griffin et al. U.S. patent application Ser. No. 10/886,766 (published as US 2005/0037964), the disclosure of which is hereby incorporated by reference.

### EXAMPLES

Examples of APC variants having reduced anticoagulant activity over endogenous APC or wild-type recombinant APC, while retaining beneficial anti-apoptotic activity are described in Griffin et al. U.S. patent application Ser. No. 10/886,766 (published as US 2005/0037964). Specific examples are variants of recombinant APC or protein C having at least one mutation at a residue in a protease domain of a surface loop selected from the group consisting of loop 37, the calcium loop, and the autolysis loop. One aspect of this embodiment comprises mutating the recombinant APC or protein C at any surface loop of the protease domain and determining the variant APC's anticoagulant and cytoprotective activities in assays as described.

To screen the candidate protein C variant for desirable properties in accordance with the invention, the protein C variant would be converted to the activated form (APC) prior to measuring activities. In another aspect of this embodiment, a library of candidate agents is selected which are variants of recombinant APC or protein C having at least one mutation at a residue in a protease domain of a surface loop selected from the group consisting of loop 37, the calcium loop, and the autolysis loop.

Such mutants of human APC, as described in Griffin et al. U.S. patent application Ser. No. 10/886,766) include RR229/230AA-APC (replacing Arg229 and Arg230 in the calcium-binding loop of APC with alanine residues), KKK191-193AAA-APC (3K3A-APC) (replacing three consecutive lysine residues in loop 37 with three alanines), and RR306/312AA-APC (replacing Arg306 and Arg312 in the autolysis loop with alanine residues).

Another human APC variant having reduced anticoagulant activity over endogenous APC or wild-type recombinant APC while retaining beneficial anti-apoptotic activity is RR229/230AA plus KKK191-193AAA-APC (designated 5A-APC) (combination of mutations of arginines 229 and 230 to alanines in the calcium-binding loop and lysines 191, 192 and 193 to alanines in loop 37).

In the human 5A-APC mutant, the two arginine residues (Arg229 and Arg230) in the calcium-binding loop of APC were replaced with two alanine residues, and three consecutive lysine residues (Lys191, Lys192, and Lys193) in loop 37 were replaced with three alanine residues. The resulting APC variant combines the alanine substitutions of Example 1 (RR229/230AA-APC) with the alanine substitutions of Example 2 (KKK191-193AAA-APC) to produce RR229/230AA plus KKK191-193AAA-APC (designated 5A-APC).

Distinguishing the cytoprotective from the anticoagulant activities of APC may be accomplished by making recombinant human wild type (wt) APC and a protease domain mutant, 5A-APC(RR229/230AA+KKK191-193AAA), a Gla domain mutant, PTGIa-APC (APC with prothrombin residues 1-46) (Smirnov et al JBC 1998) and an active site mutant, S360A-APC. Active site titration and chromogenic assays show that wtAPC, 5A-APC and PTGIa-APC have full enzymatic activity while S360A-APC has none. 5A-APC has almost no anticoagulant activity (<3%) whereas PTGIa-APC has 300% of wtAPC anticoagulant activity. Further, wtAPC and 5A-APC each bind to EPCR with similar affinity, whereas, in contrast, PTGIa-APC binds very weakly to EPCR. Anti-inflammatory and anti-apoptotic activities of the hypo-anticoagulant 5A-APC and the hyper-anticoagulant PTGIa-APC may be compared to those of wtAPC. The ability of 5A-APC to inhibit LPS-induced TNFα secretion from U937 monocytic cells is indistinguishable from that of wtAPC with half-maximum inhibition at 5.4 nM and 6.5 nM, respectively. Neither S360A-APC nor the protein C zymogen inhibit LPS-induced TNFα secretion, indicating that a functional APC active site is required. Anti-EPCR antibodies blocking APC binding prevent the anti-inflammatory activity of wtAPC, indicating binding of APC to EPCR on U937 cells is required.

The anti-apoptotic activity of each human APC species may be determined in staurosporine-induced endothelial cell apoptosis assays. Dose-dependent inhibition of apoptosis by 5A-APC is indistinguishable from that by wt-APC with half-maximum inhibition at 0.70 and 2.0 nM, respectively. In contrast to this potent anti-apoptotic activity of wtAPC and 5A-APC, the hyper-anticoagulant PTGIa-APC requires a 24-fold higher concentration for half-maximal inhibition of endothelial apoptosis. The S360A-APC will show no significant inhibition of endothelial apoptosis. Hence, the human 5A-APC variant with <3% anticoagulant activity exhibits normal anti-inflammatory and anti-apoptotic activities in vitro on monocytic and endothelial cells. These APC variants may be useful for in vivo assessment of the relative importance of APC's anticoagulant vs. cytoprotective activities. This highlights important distinctions between structural requirements for APC's anticoagulant functions compared to its anti-inflammatory and anti-apoptotic activities. These structural insights may lead to safer therapeutic APC variants that retain one or more of APC's beneficial cytoprotective effects but that have reduced bleeding risk due to reduction in anticoagulant activity.

The invention comprises several embodiments which are described below.

Prodrug embodiments of this invention may involve recombinant protein C or variants thereof that, following conversion of protein C to APC, exhibit reduced anticoagulant activity while retaining normal or near-normal cell protective activities, i.e., have a ratio of anti-apoptotic:anticoagulant activity greater than 1.0.

Therapeutic compositions comprising the variant APC of the invention may be provided in dosage form. In one aspect of this embodiment, the therapeutic compositions of the invention may further comprise a pharmaceutically acceptable carrier and may still further comprise components useful for delivering the composition to a subject's brain. Such pharmaceutical carriers and delivery components are known in the art. Addition of such carriers and other components to the composition of the invention is well within the level of skill in this art. For example, a permeable material may release its contents to the local area or a tube may direct the contents of a reservoir to a distant location of the brain.

The pharmaceutical compositions of the invention may be administered as a formulation, which is adapted for direct application to the central nervous system, or suitable for passage through the gut or blood circulation. Alternatively, pharmaceutical compositions may be added to the culture medium. In addition to active compound, such compositions may contain pharmaceutically-acceptable carriers and other ingredients known to facilitate administration and/or enhance uptake. It may be administered in a single dose or in multiple doses, which are administered at different times. A unit dose of the composition is an amount of an APC mutant that provides cytoprotection, inhibits apoptosis or cell death, and/or promotes cell survival but does not provide a clinically significant anticoagulant effect, a therapeutic level of such activity, or has at least reduced anticoagulant activity in comparison to previously described doses of activated protein C. Measurement of such values are within the skill in the art: clinical laboratories routinely determine these values with standard assays and hematologists classify them as normal or abnormal depending on the situation.

The pharmaceutical compositions of the invention may be administered by any known route. By way of example, the composition may be administered by a mucosal, pulmonary, topical, or other localized or systemic route (e.g., enteral and parenteral). In particular, achieving an effective amount of activated protein C, prodrug, or functional variant in the central nervous system may be desired. This may involve a depot injection into or surgical implant within the brain. “Parenteral” includes subcutaneous, intradermal, intramuscular, intravenous, intra-arterial, intrathecal, and other injection or infusion techniques, without limitation.

Suitable choices in amounts and timing of doses, formulation, and routes of administration can be made with the goals of achieving a favorable response in the subject (i.e., efficacy or therapeutic), and avoiding undue toxicity or other harm thereto (i.e., safety). Administration may be by bolus, brief infusion, or by continuous infusion; bolus administration is preferred. Bolus refers to administration of a drug (e.g., by injection) in a defined quantity (called a bolus) over a period of time. Continuous infusion refers to continuing substantially uninterrupted the introduction of a solution into a blood vessel for a specified period of time. A bolus generally is administered to a subject as a discrete injection or over the course of about an hour or less; a brief infusion generally occurs over the course of about 1 hour to about 4 hours; a continuous infusion generally occurs over the course of more than about 4 hours.

A bolus of the formulation administered only once to a subject is a convenient dosing schedule. Preferably, treatment involves giving a subject one bolus administration, or two or more bolus administrations in a time period of about 3-12 hours. Preferably, treatment involves giving a subject two or more bolus administrations in a time period of about 3-10 hours. Administrations may be given four times daily, three times daily, twice daily, once daily, every other day, every three days, every four days, every five days, once a week, every 10 days, or once a month. In one embodiment, one bolus administration is given to a subject each day for seven days.

A dose of activated protein C, activated protein C variant, or protein C variant may be provided as a single bolus, by a single brief infusion, or by continuous infusion. A dose may be given each time over multiple bolus administrations or by multiple brief infusions. In one embodiment, the dose is provided as a brief infusion over the course of about 1 hour to about 4 hours. In another embodiment, the dose is provided as a single bolus over the course of about an hour or less. In another embodiment, the dose is provided as a single bolus over the course of about 20 minutes. In one embodiment the dose is provided as a single bolus in a discrete injection. In another embodiment, the dose is provided by continuous infusion. The duration of administration of a therapeutic amount of activated protein C or activated protein C variant may be tailored to best suit the subject's needs.

A dose of activated protein C, activated protein C variant, or protein C variant may be administered to a patient as a course of treatment based upon the patient, symptoms, disease, or other factors. A dose may be administered one or more times per day for one or more days. In one embodiment, a dose is provided as a single bolus each day over the course of seven days. In another embodiment, a dose is provided as a bolus administration every 12 hours for 96 hours. In another embodiment, a dose is provided as a bolus administration every 24 hours for 96 hours. In yet another embodiment, a dose is provided by continuous infusion from between 24 to 168 hours. In another embodiment, a dose is provided by continuous infusion over 96 hours.

A dose of activated protein C, activated protein C variant, or protein C variant may be expressed as mg of ingredient per kg of a subject's body mass, that is, mg/kg. Preferred doses for administration are 16 mg/kg or less, 8 mg/kg or less, 4 mg/kg or less, 2 mg/kg or less, 1.6 mg/kg or less, 1 mg/kg or less, 0.8 mg/kg or less, 0.6 mg/kg or less, 0.5 mg/kg or less, 0.4 mg/kg or less, 0.33 mg/kg or less, 0.2 mg/kg or less, 0.1 mg/kg or less, 0.08 mg/kg or less, 0.067 mg/kg or less, 0.05 mg/kg or less, 0.04 mg/kg or less, 0.03 mg/kg or less, 0.02 mg/kg or less, 0.01 mg/kg or less, 0.005 mg/kg or less, depending on the species of the subject or disease to be treated.

Dosage levels of active ingredients in a pharmaceutical composition can also be varied so as to achieve a transient or sustained concentration of the compound or derivative thereof in a subject and to result in the desired therapeutic response. The frequency of administration of activated protein C variant or protein C variant may be tailored to best suit the subject's needs.

Thus, “therapeutic” refers to such choices that involve routine manipulation of conditions to achieve a desired effect (e.g., inhibition of apoptosis or cell death, promotion of cell survival, cytoprotection, neuroprotection, or combinations thereof). The amount of mutant protein C or mutant activated protein C administered to subjects may be higher than doses of recombinant protein C or activated protein C, if necessary for maximal cytoprotection, because of the reduced risk of bleeding or serious bleeding. In this manner, “therapeutic amount” refers to the total amount of activated protein C variant or protein C variant that achieves the desired cytoprotective effect, but with reduced risk for bleeding due to reduced anticoagulant activity (for bolus administration, e.g., 16 mg/kg or less, 8 mg/kg or less, 4 mg/kg or less, 2 mg/kg or less, 1.6 mg/kg or less, 1 mg/kg or less, 0.8 mg/kg or less, 0.6 mg/kg or less, 0.5 mg/kg or less, 0.4 mg/kg or less, 0.33 mg/kg or less, 0.2 mg/kg or less, 0.1 mg/kg or less, 0.08 mg/kg or less, 0.067 mg/kg or less, 0.05 mg/kg or less, 0.04 mg/kg or less, 0.03 mg/kg or less, 0.02 mg/kg or less, 0.01 mg/kg or less, 0.005 mg/kg or less, depending on the species of the subject or disease to be treated).

The therapeutic amount may be about 0.005 mg to about 16 mg per kilogram of a subject's body mass. More particularly, the therapeutic amount may be about 0.01 mg to about 1.6 mg per kilogram of a subject's body mass. More particularly, the therapeutic amount may be about 0.03 mg to about 0.8 mg per kilogram of a subject's body mass. Preferably, the therapeutic amount may be about 0.067 mg to about 0.33 mg per kilogram of a subject's body mass. Preferably, the therapeutic amount may be about 0.06 mg to about 0.4 mg per kilogram of a subject's body mass administered as a single bolus. Preferably, the therapeutic dose is administered by bolus at about 0.07 mg to about 0.33 mg per kilogram of a subject's body mass. Preferably, the therapeutic dose is administered by bolus over the course of about 20 minutes at about 0.067 mg to about 0.33 mg per kilogram of a subject's body mass.

A therapeutic amount may be provided as a single bolus, by a single brief infusion, or by continuous infusion. A therapeutic amount may be given each time over multiple bolus administrations or by multiple brief infusions. In one embodiment, the therapeutic amount is provided as a brief infusion over the course of about 1 hour to about 4 hours. In another embodiment, the amount is provided as a single bolus over the course of about an hour or less. In another embodiment, the amount is provided as a single bolus over the course of about 20 minutes. In one embodiment the amount is provided as a single bolus in a discrete injection. In another embodiment, the amount is provided by continuous infusion. The duration of administration of a therapeutic amount of activated protein C or activated protein C variant may be tailored to best suit the subject's needs.

Therapeutic amounts may be administered to a patient as a course of treatment based upon the patient, symptoms, disease, or other factors. A therapeutic amount may be administered one or more times per day for one or more days. In one embodiment, a therapeutic amount is provided as a single bolus each day over the course of seven days. In another embodiment, a therapeutic amount is provided as a bolus administration every 12 hours for 96 hours. In another embodiment, a therapeutic amount is provided as a bolus administration every 24 hours for 96 hours. In yet another embodiment, a therapeutic amount is provided by continuous infusion from between 24 to 168 hours. In another embodiment, a therapeutic amount is provided by continuous infusion over 96 hours.

In one embodiment, two therapeutic amounts are provided about 3-10 hours apart from one another. In one embodiment, a therapeutic amount is provided as a first bolus administration followed by a second bolus administration of a therapeutic amount 7 hours later. In one embodiment, a therapeutic amount is provided as a first bolus administration followed by a second bolus administration of a therapeutic amount 10 hours later. In another embodiment, a therapeutic amount is provided as a bolus every 12 hours.

The therapeutic amount may be based on titering to a blood level amount of APC of about 0.01 μg/ml to about 1.6 μg/ml, or from about 0.01 μg/ml to about 0.5 μg/ml. It is also within the skill of the art to start doses at levels lower than required to achieve the desired therapeutic effect and to gradually increase the dosage until the desired effect is achieved. It is likewise within the skill of the art to determine optimal concentrations of variants to achieve the desired effects in the in vitro and ex vivo preparations of the invention, e.g., about 1-100 nM.

Screening candidate agents to identify other variants of recombinant APC having therapeutic potential in accordance with the invention is within the ordinary skill in the art. Examples include using methods of inducing apoptosis and other tests for measuring apoptotic activity and anticoagulant activity.

Protein C Activation

Recombinant forms of protein C can be produced with a selected chemical structure (e.g., native, mutant, or polymorphic). As an illustration, a gene encoding human protein C is described in U.S. Pat. No. 4,775,624 and can be used to produce recombinant human protein C as described in U.S. Pat. No. 4,981,952. Human protein C can be recombinantly produced in tissue culture and activated as described in U.S. Pat. No. 6,037,322. Natural human protein C can be purified from plasma, activated, and assayed as described in U.S. Pat. No. 5,084,274. The nucleotide and amino acid sequence disclosed in these patents may be used as a reference for protein C.

Recombinant wild-type APC (wt-APC), RR229/230AA-APC (229/230-APC), KKK191/192/193AAA-APC (3K3A-APC), and RR229/230AA plus KKK191/192/193AAA-APC (5A-APC) may be prepared as described (Gale et al., supra, 1997; Gale et al., supra, 2000; Gale et al., supra, 2002).

Protein C will be activated by thrombin (3281 U/mg, Enzyme Research Labs, South Bend, Ind.). Protein C in HBS (HEPES buffered saline, 50 mM HEPES, 150 mM NaCl) with 2 mM EDTA and 0.5% bovine serum albumin (BSA), pH 7.4, at a concentration of 600 μg/mL will be incubated for 2.5 hours with 12 μg/mL thrombin at 37° C., followed by the addition of 1.1 units of hirudin (Sigma, St Louis, Mo.) per unit of thrombin to inactivate the thrombin. Control assays will be done using amidolytic assays, APTT clotting assays and FVa inactivation assays to verify that the thrombin and hirudin used had no effect on subsequent assays.

A “mutation” refers to one or more changes in the sequence of polynucleotides and polypeptides as compared to native activated protein C, and has at least one function that is more active or less active, an existing function that is changed or absent, a novel function that is not naturally present, or combinations thereof.

In summary, three examples of the variants of recombinant APC mutants of this invention, namely KKK191-193AAA-APC, RR229/230AA-APC, and RR229/230AA plus KKK191-193AAA-APC (5A-APC) are provided, that have substantial reductions in anticoagulant activity but that retain normal or near-normal levels of anti-apoptotic activity. The invention encompasses APC variants such as these, which have the highly desirable property of a high ratio of anti-apoptotic to anticoagulant activity. The invention further encompasses variants having more modest, yet still beneficial, ratios of anti-apoptotic to anticoagulant activity; such variants also would be expected to be cytoprotective while having significantly reduced risk of bleeding. The invention is not limited to variants of APC, but also includes protein C mutants which are capable of yielding desirable APC mutants, i.e., those that would have the same desirable activity ratios. The invention also is not limited to mutations on loop 37, calcium loop, or autolysis loop; the invention encompasses mutations of residues on other surface loops of the protease domain that produce the desired cytoprotective to anticoagulant ratio. Thus, APC and protein C variants of the invention are expected to be useful for therapy for subjects who will benefit from APC protective activities that are independent of APC's anticoagulant activity. Subjects would include patients at risk of damage from apoptosis to blood vessels or tissue in various organs. More specifically, but not exclusively, these subjects will include, for example, those suffering severe sepsis, ischemia/reperfusion injury, ischemic stroke, acute myocardial infarction, acute or chronic neurodegenerative diseases and organ transplantation, among other conditions.

In Vitro Experiment with Human 5-APC Mutant

Methods

The protein C expression vector for 5A-APC was constructed as described. (Gale et al., J. Biol. Chem. 277:28836-28840, 2002) Mutations (R229A+R230A+K191A+K192A+K193A) were introduced using Quickchange mutagensis (Stratagene, Cedar Creek, Tex.). Sequence analysis confirmed the presence of the mutations.

The human protein C variant containing 5 alanine substitutions (R229A+R230A+K191A+K192A+K193A) (5A-APC) was expressed using K293 cells as described (Gale et al., J. Biol. Chem. 277:28836-28840, 2002; Gale et al., Blood, 96:585-593, 2000; Gale et al., Protein Sci., 6:132-140, 1997). Purified recombinant 5A-protein C was prepared as described using a fast flow Q-Sepharose column (Gale et al., J. Biol. Chem. 277:28836-28840, 2002; Gale et al., Blood, 96:585-593, 2000; Gale et al., Protein Sci., 6:132-140, 1997).

5A-APC(R229A+R230A+K191A+K192A+K193A) was generated from 5A-protein C by thrombin activation as described (Gale et al., J. Biol. Chem. 277:28836-28840, 2002; Gale et al., Blood, 96:585-593, 2000; Gale et al., Protein Sci., 6:132-140, 1997). 5A-APC concentration was determined by active site titration according to Chase and Shaw (Biochem. Biophys. Res. Commun., 29:508-514, 1967).

Assays

Amidolytic (S-2366) assays were performed as described (Gale et al., J. Biol. Chem. 277:28836-28840, 2002; Gale et al., Blood, 96:585-593, 2000; Gale et al., Protein Sci., 6:132-140, 1997). APTT clotting time assays were performed. APC's cytoprotective effects were determined in assays of staurosporine-induced endothelial cell (EA.hy926) apoptosis as described (Mosnier and Griffin, Biochem. J., 373:65-70, 2003).

Immortalized human monocytes (U937, ATCC (Manassas, Va.)) were cultured in RPMI-based growth medium supplemented with 10% heat inactivated fetal bovine serum, penicillin (1 unit/mL), streptomycin (1 μg/mL) and glutamine (292 μg/mL). Cells subjected to experiments were between their 4^{th} to 10^{th} passage. Before each experiment, cell viability was examined by Trypan Blue staining and confirmed to be more than 99%.

Results

As noted above, 229/230-APC and 3K3A-APC have reduced anticoagulant activity whereas the anti-apoptotic activity of these variants remains essentially unaffected (Mosnier et al., Blood. 104:1740-1745, 2004). Although the anticoagulant activity of these APC variants (229/230-APC and 3K3A-APC) was reduced, they did retain detectable anticoagulant activity in APTT clotting assays. Thus, we made the APC variant in which the above mentioned mutations were combined (5A-APC with R229A+R230A+K191A+K192A+K193A). This 5A-APC has severely reduced anticoagulant activity compared to recombinant wild type APC (rwt-APC) while at least some of the direct protective effects on cells are essentially unaffected and indistinguishable from rwt-APC.

Under the conditions employed, both rwt-APC and 5A-APC had similar amidolytic activity. In contrast, S360A-APC, an APC variant with the active site Ser residue mutated to Ala, had no detectable amidolytic activity. APTT clotting assays revealed that the 5A-APC anticoagulant activity was severely reduced (<3% compared to rwt-APC defined as 100% under the conditions employed). This marks a significant reduction compared to the previously described 229/230-APC and 3K3A-APC with 13 and 5% anticoagulant activity under similar conditions, respectively.

To evaluate the activity of 5A-APC for direct protective effects on cells, both APC anti-inflammatory activity on monocytes and APC anti-apoptotic activity on endothelial cells were analyzed. APC anti-inflammatory activity was determined by its ability to inhibit lipopolysaccharide (LPS)-induced cytokine release from monocytes (U937 cells). Both rwt-APC and 5A-APC inhibited LPS-induced tumor necrosis factor α (TNFα) release from monocytes. Dose response titrations of rwt-APC and 5A-APC indicated that the relative potency of rwt-APC and 5A-APC were indistinguishable. Similar results were obtained for analysis of inhibition of LPS-induced interleukin 6 (IL-6) release from monocytes. Both rwt-APC and 5A-APC inhibited dose-dependently LPS-induced IL-6 release from monocytes. No differences were observed between the concentrations of rwt-APC and 5A-APC required to achieve half-maximal inhibition of LPS-induced IL-6 release. These results indicate that 5A-APC has normal APC anti-inflammatory activity compared to rwt-APC.

APC anti-apoptotic activity was determined in assays of staurosporine-induced endothelial cell (EA.hy926) apoptosis as described (Mosnier and Griffin, Biochem. J., 373:65-70, 2003; Mosnier et al., Blood. 104:1740-1745, 2004). Both rwt-APC and 5A-APC inhibit staurosporine-induced endothelial cell apoptosis. The concentrations of rwt-APC and 5A-APC required to achieve half-maximal inhibition of apoptosis were indistinguishable as determined by dose response titrations of rwt-APC and 5A-APC. Therefore, 5A-APC has essentially normal anti-apoptotic activity compared to rwt-APC, although its anticoagulant activity is severely decreased.

APC variants such as these are useful for therapy for subjects who will benefit from APC protective activities that are independent of APL's anticoagulant activity. Subjects who can benefit from therapy with such APC variants include subjects at risk of damage to blood vessels or tissue in various organs caused, at least in part, by apoptosis. These subjects will include, for example, animals or humans suffering sepsis or severe sepsis, ischemia/reperfusion injury, stroke, ischemic stroke, acute myocardial infarction, acute or chronic neurodegenerative diseases, radiation damage or those undergoing organ transplantation or chemotherapy, among other conditions.

Recombinant human APC variants were added to human plasma, and the loss of APC enzymatic activity was monitored using standard amidolytic assays. The half-lives in human plasma, for the recombinant human wt APC and APC variants were similar, indicating that the cause of loss of anticoagulant activity of APC variants is not a decreased half-life.

In Vivo Experiments with Murine Wild Type APC and APC Mutants

Protective effect of activated protein C in murine endotoxemia:

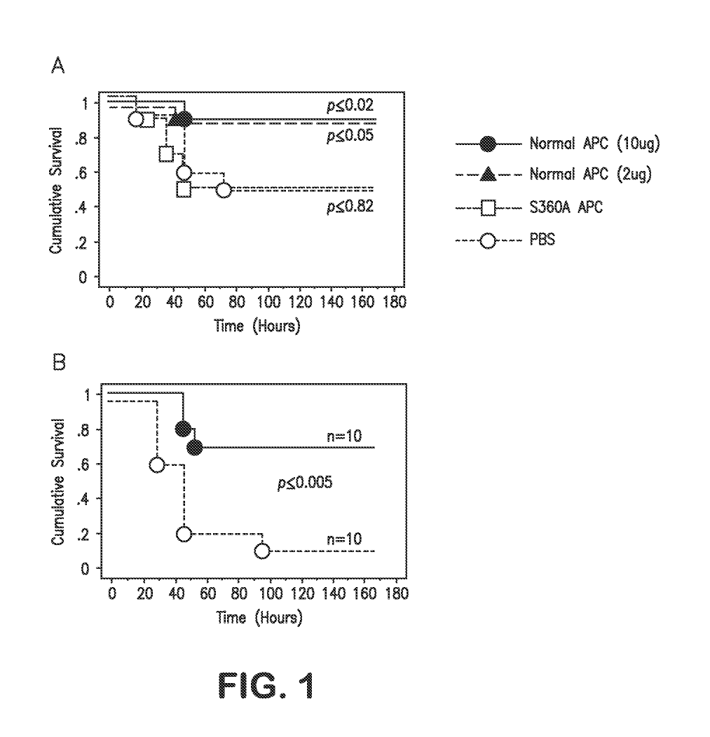

Intravenous administration of 10 or 2 μg of recombinant murine wild type (wt) APC over a 20 min time period into male 057BI/6 mice (28-32 g bodyweight), administered concomitant with a LD50 or LD90 i.p. dose of E. coli LPS, significantly reduced 7-day mortality (FIG. 1A, B). As judged by visual observation, administration of APC prevented the reduction of locomotor activity and general sickly appearance seen as early as ˜2 h after LPS administration in LPS-treated mice receiving carrier solution only. Administration of 10 μg recombinant murine S360A-APC, which possesses minimal proteolytic activity due to substitution of serine 360 by alanine at the reactive center of the catalytic triad did not reduce mortality (FIG. 1A). Thus, the therapeutic efficacy of APC requires full proteolytic activity. A bolus infusion of 10 or 2 μg of APC into the retro-orbital venous plexus at the time of LPS administration was as effective in reducing 7-day mortality as administration via a 20 min i.v. infusion of the same amount of APC.

Thus, a single dose of recombinant murine APC of 0.33 or 0.067 mg/kg, administered at the onset of LPS-induced endotoxemia, reduces 7-day mortality.

APC Reduces Tissue Damage and Preserves Vascular Integrity

To examine the effects of APC treatment on the pre-mortem pathology of endotoxemia, a cohort of animals receiving an LD50 dose of LPS concomitant with either 10 μg APC, 10 μg of S360A-APC, or carrier solution (PBS) was sacrificed 24 hours after induction of endotoxemia. At this time point, ≧90% of animals in all experimental groups were still alive, but death occurs shortly thereafter in the control group treated with S360A-APC (see FIG. 1A). Based on the above data, 9 of 10 APC-treated animals, and 5 of 10 animals treated with S360A-APC are predicted to survive.

LPS exposure results in mice in a marked activation of the coagulation and fibrinolytic systems, reflected in increased plasma levels of thrombin-antithrombin (TAT) complex and D-dimer fibrin degradation product, and platelet consumption; stimulation of inflammatory cytokine responses, margination of leukocytes reflected in reduced numbers of circulating lymphocytes and monocytes, and increased numbers of granulocytes.

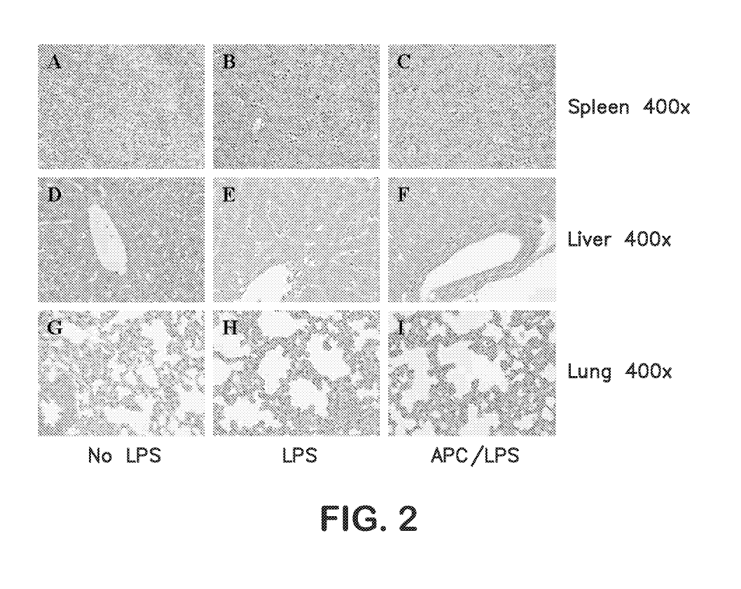

No significant correlation with APC treatment was measured for TAT and platelet consumption. APC treatment but not S360A-APC treatment was associated with reduced circulating levels of D-dimer. Differential blood cell counts, histopathology of lung, liver, and spleen (FIG. 2), and composition of the immune cell population in the spleen were not affected by APC treatment.

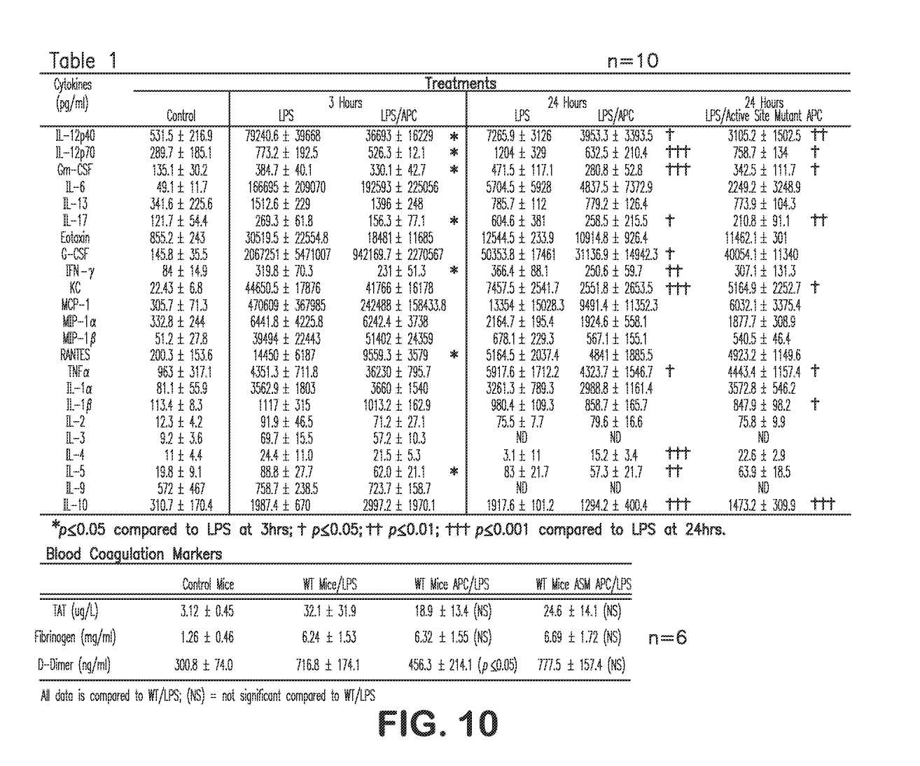

At the 24 h time point, the mean plasma level of 19 of the 23 cytokines assayed was lower in the APC-treated group, but statistically significant correlations with APC treatment were found only for IL-12p40/IL-12p70, GM-CSF, and KC, (see 1 at FIG. 10). However, similar reductions in the levels of these cytokines were measured in mice receiving the therapeutically ineffective S360A-APC.

To determine the effect of APC on the early cytokine response and to correlate observed changes with overall outcome, blood samples were drawn from a second cohort of mice 3 hours after LPS challenge, and correlated with 7-day survival. 10 mice received APC and 10 mice (controls) received only vehicle. In this experiment, 8 of 10 APC-treated mice and 1 of 10 PBS-treated mice survived. Significant correlation with treatment and survival was observed for IL-12p40, Eotaxin, RANTES, TNFα, and IL-2, measured at the 3 hour time point.

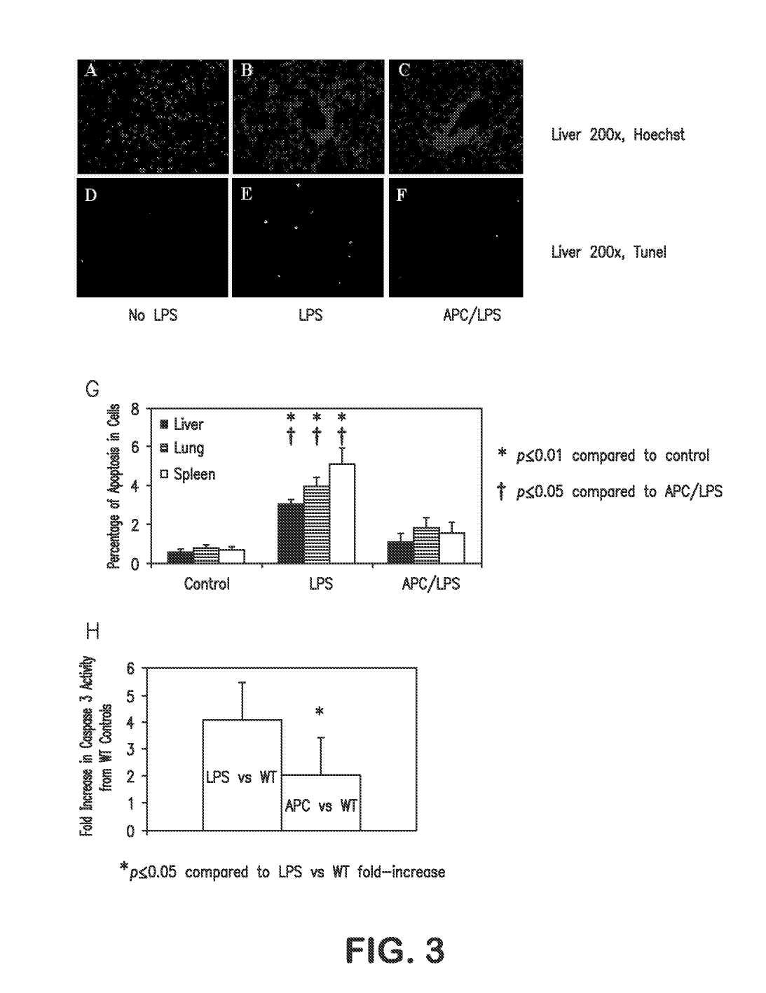

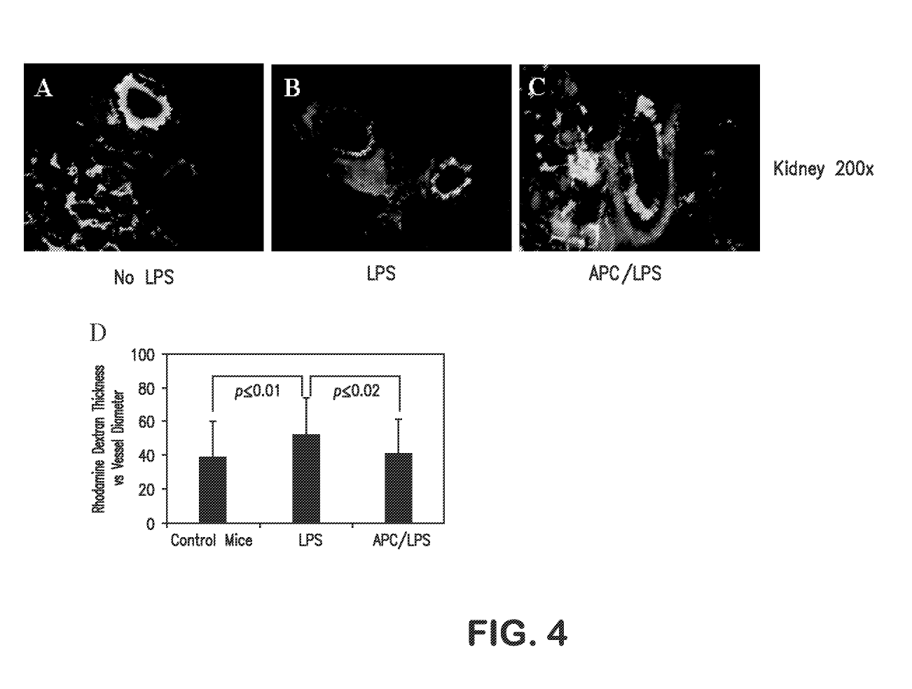

Treatment with APC, but not with S360A-APC, reduced the number of apoptotic cells in tissue sections of lung, spleen, and liver as detected by TUNEL stain (FIG. 3). In the liver, this correlated with a suppression of caspase 3 activity in whole tissue extracts. The predominant cell type, i.e. endothelial versus non-endothelial, undergoing apoptosis in endotoxemia could not be determined in our study. APC treatment also preserved vascular endothelial barrier function in the kidney, as demonstrated by measuring the permeability of the vascular bed for fluorescent tracers of different molecular weight (FIG. 4).

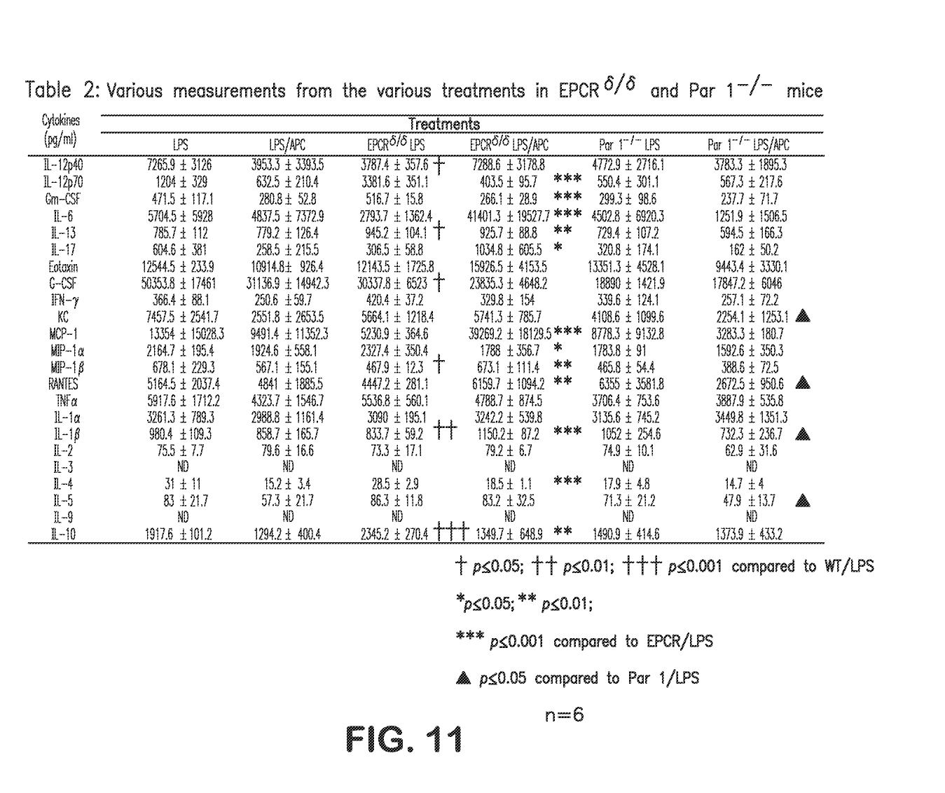

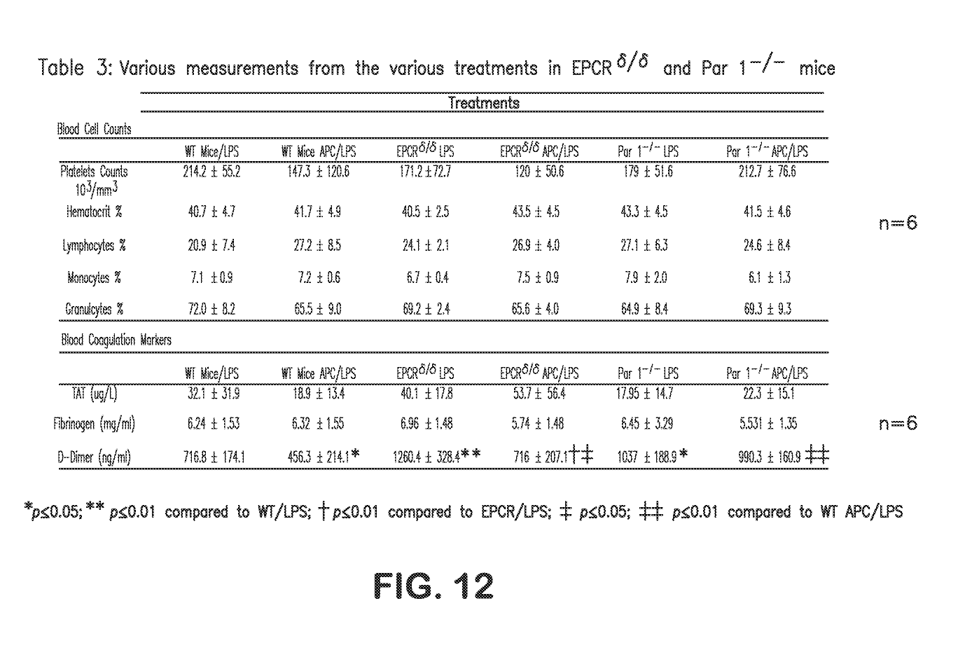

These analyses did not establish a clear correlation between levels of IL-6, INFγ, TNFα, IL-1, MCP-1, which have been associated with potential APC effects in the setting of endotoxemia. On the other hand, we document a beneficial effect of APC treatment on kidney vascular integrity and on the extent of tissue damage associated with apoptosis in lung, spleen, and liver. See FIGS. 3-4 and also Tables 1-3 (FIGS. 10-12).

Therapeutic Efficacy of APC Requires Functional EPCR and PAR 1

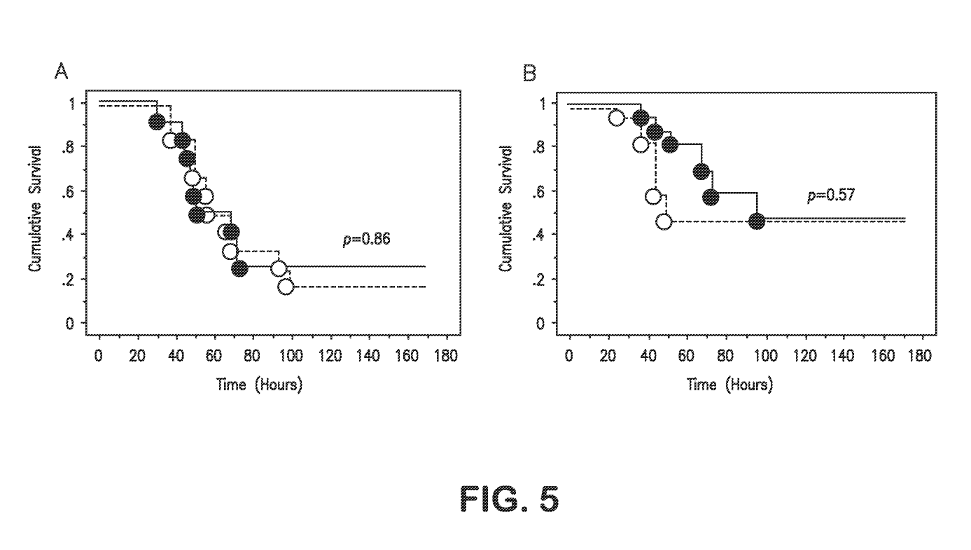

To investigate whether APC reduces mortality through interaction with the endothelial protein C receptor (EPCR) and/or protease activated receptor-1 (PAR1), the therapeutic efficacy of APC was determined in genetically altered mice expressing greatly diminished amounts of EPCR (EPCIR^{∂/∂}, less than 10% of normal EPCR expression), and in mice completely devoid of PAR1 (PAR1^{-/-}) (FIG. 5 A, B). Mortality and survival time of EPCIR^{∂/∂} and PAR1^{-/-} mice after challenge with LPS was indistinguishable from that of wild type mice in the absence of APC treatment. Treatment of EPCIR^{∂/∂} mice with APC did not alter survival time or 7-day mortality, compared to vehicle treated animals. APC treatment somewhat prolonged survival time in PAR1^{-/-} mice, but did not affect 7-day mortality. Also, in PAR1^{-/-} mice, based on visual observations, APC treatment did not prevent the characteristic behavioral changes induced by LPS.

In the absence of APC treatment, TAT levels and platelet consumption, measured 24 h after LPS challenge, in PAR1^{-/-} and in EPCIR^{∂/∂} mice were not different from those measured in wild type mice, and 24 h D-dimer levels were significantly increased in EPCIR^{∂/∂} and in PAR1^{-/-} mice, as compared to wild type mice. Compared to wild type mice, APC treatment reduced D-dimer levels to a similar extent in EPCIR^{∂/∂} mice, but not in PAR1^{-/-} mice.

Without APC treatment, the cytokine profile of LPS-challenged EPCIR^{∂/∂} mice, as measured at the 24 h time point, was similar to that of wild type mice, with moderate reductions of IL-12p40, G-CSF, MIP-1β, and IL-1β, slight increases of GM-CSF and IL-13, and a significant augmentation of IL-10 (P<0.001). APC treatment of LPS-challenged EPCIR^{∂/∂} mice decreased with borderline significance the levels of IL-12p70 and GM-CSF, reproducing the effect of APC in wild type mice. In contrast to wild type mice, APC treatment of EPCIR^{∂/∂} mice elicited a 10-fold and 7-fold augmentation of IL-6 and MCP-1 levels, respectively.

The cytokine profile of LPS-challenged PAR1^{-/-} mice, as measured at the 24 h time point, was similar to that of wild type mice, with the exception of G-CSF, which was significantly diminished (P<0.001). APC treatment of LPS-challenged PAR1^{-/-} mice produced significant reductions of IL-6, KC, MCP-1, and RANTES, in comparison to PBS-treated PAR1^{-/-} controls (table 2).

Generation of Recombinant Murine APC Variants with Reduced Anticoagulant, but Preserved Signaling Function

Previous work demonstrated that alanine substitutions in two surface loops of human APC diminish its anticoagulant activity while preserving EPCR- and PAR1-dependent signaling functions (Mosnier et al., Blood. 104:1740-1745, 2004). Here, corresponding variants of recombinant murine APC were prepared, i.e. (i) RR230/231AA-APC, (ii) 3K3A-APC (KKK192/193/194AAA), and (iii) 5A-APC, combining the RR230/231AA and 3K3A substitutions, and tested for loss of anticoagulant function.

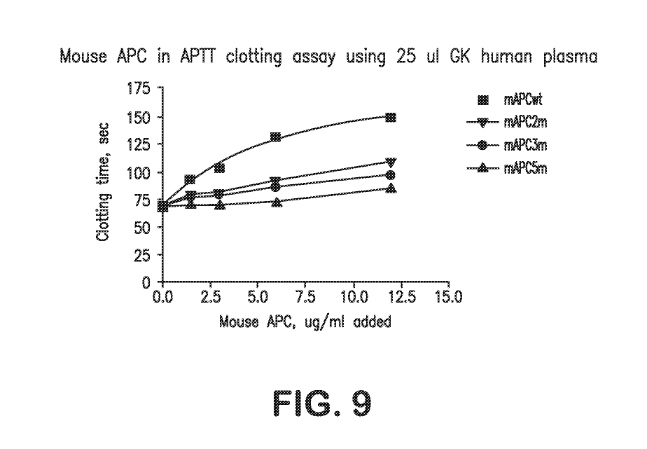

Recombinant wild type mouse APC, but not catalytically inactive S360A-APC significantly prolonged the clotting time of mouse plasma in an APTT-assay format that measures the ability of APC to prolong clotting time due to inactivation of endogenous fV/fVa. All other APC variants exhibited reduced, but not absent anticoagulant activity. The anticoagulant activity of murine APC variants compared to that of wild type (wt) murine APC, as shown in FIG. 9.

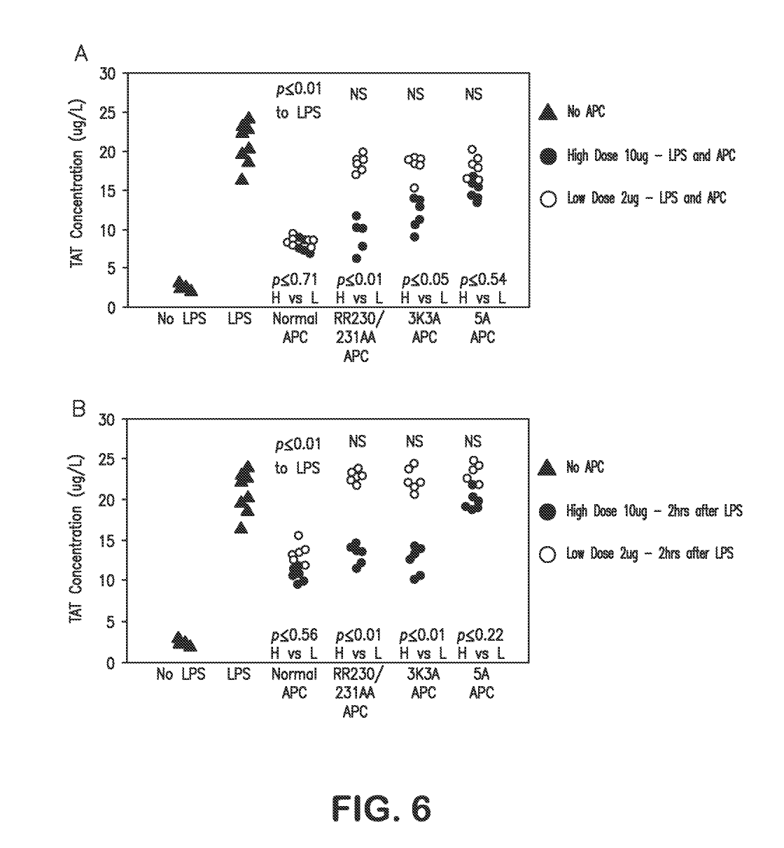

The anticoagulant activity of APC variants was tested in vivo under conditions of LPS-induced endotoxemia. TAT complex was measured and taken as a measurement of thrombin generation. The results shown in FIG. 6A demonstrate that a 10 μg bolus of wild type APC, along with LPS injection 5 min after initiation of APC infusion, elicits a suppression of circulating TAT complex, as determined 2 hours after LPS challenge; the same dose of 5A-APC produces a markedly diminished anticoagulant effect at 2 hours. RR230/231AA-APC and 3K3A-APC show intermediate reductions of TAT complex levels and have less in vivo anticoagulant activity than normal (wild type) APC. A 5-fold lower dose (2 μg) of each APC species demonstrated persistent anticoagulation by wild type APC, reduced anticoagulation by RR230/231AA-APC and 3K3A-APC, and essentially an absence of anticoagulation by 5A-APC, as judged by levels of TAT complex. To account for altered half-life or clearance of APC mutants as the possible cause of diminished anticoagulant activity, APC was administered 2 hours after LPS-challenge, and TAT levels were determined 30 min after APC administration (FIG. 6B). In these experiments, the 10 μg bolus of wild type APC showed anticoagulant potency similar to RR230/231AA-APC and 3K3A-APC, whereas 5A-APC showed almost no anticoagulant activity (FIG. 6B). In these latter experiments (FIG. 6B), although the 2 μg dose of wild type APC was anticoagulant, the 2 μg doses of to RR230/231AA-APC, 3K3A-APC, and 5A-APC displayed minimal in vivo anticoagulant activity under these experimental conditions of endotoxemia.

Comparable Efficacy of Variant APC and Wild Type APC in Endotoxemia

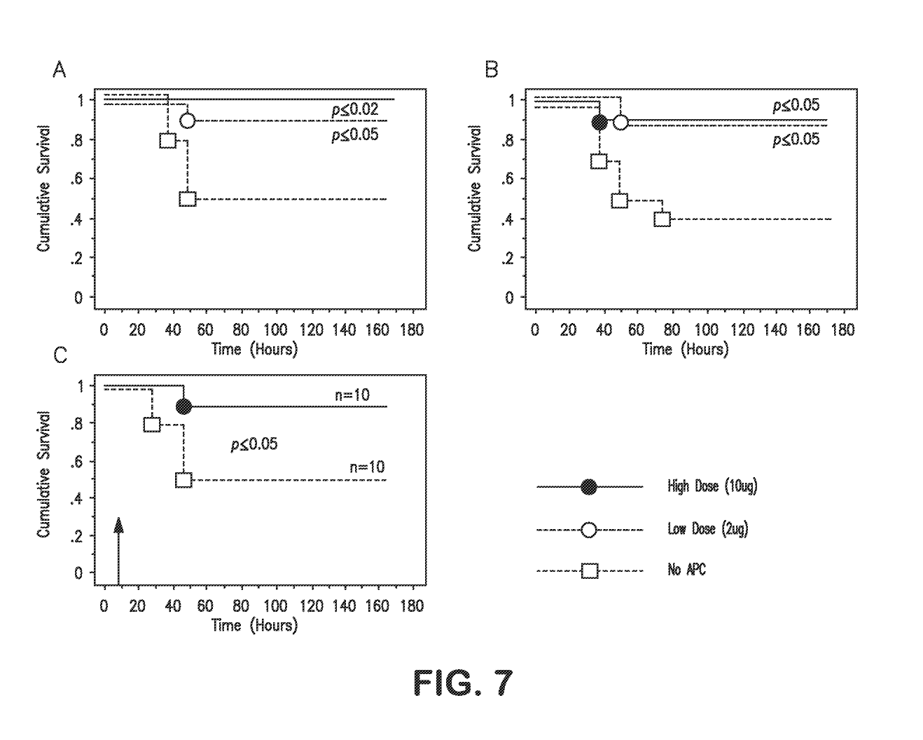

Next, we compared the ability of APC variants with reduced (RR230/231AA-APC) or greatly reduced (5A-APC) anticoagulant activity to the therapeutic efficacy of wild type APC in reducing mortality of LPS challenged mice. At both high (10 μg) and low (2 μg) doses, all APC variants significantly reduced 7-day mortality (FIG. 7 A, B) when APC was administered early. 5A-APC administered as a 2 μg bolus was effective in preventing death when given 3 hours after LPS challenge, i.e., when given at a time when endotoxemia was already established in the animal (FIG. 7 C). These latter data demonstrate that a more than 90% reduction of APC's anticoagulant activity does not diminish its therapeutic efficacy in lethal murine endotoxemia and that an APC variant can reduce mortality even after sepsis is established.

Therapeutic Efficacy of 5A-APC and Wild Type APC in Lethal Bacterial Sepsis

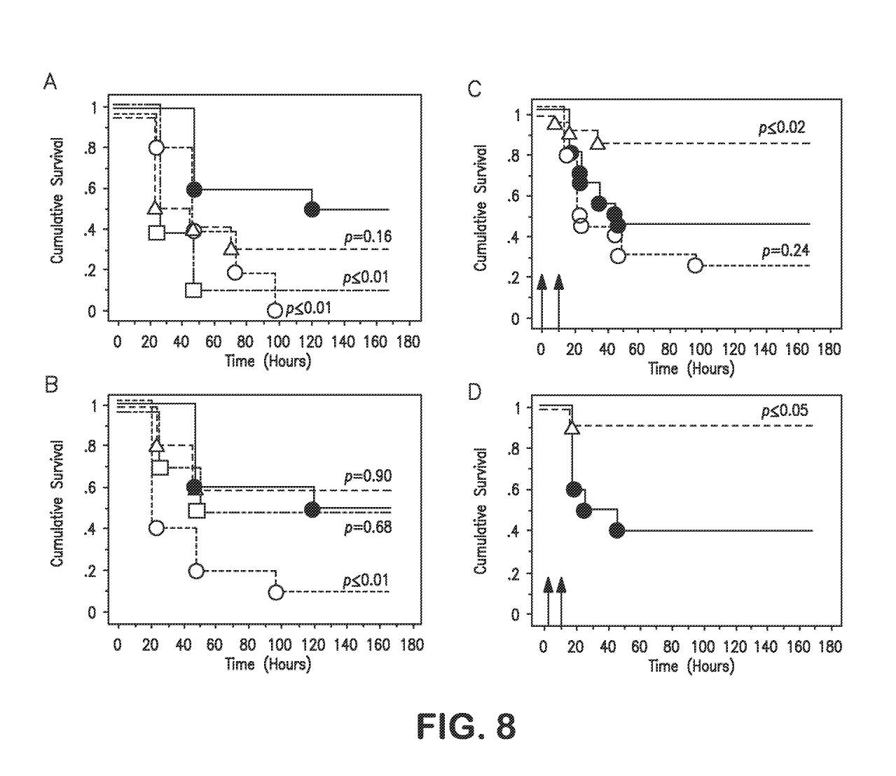

The potential therapeutic efficacy of APC and variants thereof was investigated in an animal model of bacterial sepsis induced by inoculating mice i.p. with an LD50 of live Staphylococcus aureus. Wild type APC administered as a single i.v. bolus at 10 μg or 2 μg at the time of bacterial inoculation, significantly increased mortality (FIG. 8A, 8B). In contrast, 5A-APC neither worsened nor improved outcome at either dose (FIG. 8A, 8B). Administering two consecutive 2 μg doses of wild type APC (first dose at the time of inoculation and second dose given at 10 h thereafter, before the onset of mortality) did not compromise survival, but did not improve mortality, compared to PBS-treated controls. In contrast, 5A-APC significantly reduced mortality following the same regimen of administering the APC variant at 0 hours and 10 hours (FIG. 8C). A similar strong benefit of 5A-APC for reducing mortality was documented when the first dose of 5A-APC was administered at 3 h followed by a second dose at 10 h after bacterial inoculation, i.e. when both doses were given after the establishment of sepsis (FIG. 8D).

Delaying the first dose of APC until 3 hours after bacterial inoculation, i.e. until after the initial clearance of S. aureus from the peritoneal cavity has occurred, followed by a second dose at 10 hours, may be particularly beneficial to the subject. In general, 5A-APC and APC will be more effective in settings where bacterial growth is controlled; on the other hand, the deleterious effects observed with APC are greatly diminished when using 5A-APC.

These findings show that 5A-APC with near absent anticoagulant activity is significantly more effective than wild type APC in reducing mortality in bacterial sepsis, even when administered after disease onset or only after sepsis onset. This might arise, at least in part, because the anticoagulant activity of APC interferes with fibrin-dependent clearance of bacteria from the site of infection.

Materials and Methods

Animals. Male C57BI/6 mice (8-12 weeks) were used for infusion studies. Transgenic EPCR^{δ/δ} and PAR1^{-/-} male mice (8-12 weeks) were also used for both infusion studies. The experiments were performed in adherence to the National Institutes of Health guidelines on the use of laboratory animals and approved by the Medical College of Wisconsin's Institutional Animal Care and Use Committee.

Chemicals and reagents. Cytokine assays were performed using the Bio-Plex system (Bio-Rad). Enzygnost TAT Micro (Dade Berhing) was used for the TAT assays. Asserachrome (Dignostica Stago) was used for the measurement of D-Dimer. Heparin sodium salt and lipopolysaccharide (E. coli O55:B5) was purchased from Sigma Chemical Company (St. Louis, Mo.).

Data analysis. All values are expressed as mean±SD of mice/experiment. The differences between all groups were analyzed by the Student t test. Survival curves were analyzed by the Kaplan-Meyer log-rank test. All statistics were performed using the StatView (Version 5.0, SAS Institute Inc.) program for windows.

Infusion model of mice with APC. 8-12 week old C57BI/6 male mice (Charles River) were used for the infusion process. Mice were weighed to determine amount of LPS dose (40 mg/kg). Mice were initially gas-sedated in a plastic-enclosed chamber with isoflurane-soaked gauze. The mice will then be placed supine with a nose-cone, receiving continuous gas flow from a vaporizer-anesthetic machine, using 2% isoflurane mixed with oxygen. Waste/exhaled gas will be scavenged through charcoal-filled canister by vacuum line. Procedures were done in a fume hood to assure adequate exhaust of untrapped gases.

Under sterile conditions, either the femoral vein or the external jugular vein was exposed. A 0.6-mm outer-diameter silicone catheter was inserted into the vein and ligated in place. In an acute-infusion series, the catheter will be connected to a syringe injector and the recombinant Activated Protein C (APC), various APC mutants or vehicle (PBS) was infused at a rate of 6 microliters per minute for 30 minutes (total, 180 microliters). The catheter was removed, followed by ligating the vein closed. The skin wound was then closed and the mouse was immediately removed from inhalation anesthesia to achieve rapid resuscitation. The mouse was then given the LPS via an intra-peritoneal (ip) injection.

In similar experiments, mice were given a bolus dose of either APC, one of the various APC mutants or PBS through an intra-venous retro-orbital injection. Mice were anesthetized with 2.5% avertin then given the i.v. injection. Mice were then given an i.p. dose of the 40 mg/kg dose of LPS. Mouse were then allowed to recovered and observed for survival over a 7-day period.

Analysis of coagulation in mouse plasma samples taken 2 hours after LPS. Mice were divided into two groups, and the first group given with APC at the same time as receiving LPS while the second group was given APC 2 hours after receiving LPS. In the first group, mice were infused with recombinant APC or various APC mutants at either 10 μg or 2 μg as described above. Mice were given LPS via i.p. injection then allowed to rest for 2 hours and given 500 U of heparin. Blood was then removed through a vena cava blood draw. Plasma was removed from the blood by centrifugation at 2,000×g for 20 minutes. Plasma was then flash frozen and stored at −80° C. In the second group, mice received LPS via an i.p. injection then rested for 2 h. Mice were then given recombinant APC or an APC mutant for 20 minutes. Mice were then given 500 U of heparin and blood was removed through a vena cava draw. Plasma was isolated as described above and stored at −80° C. Once all plasma was collected it was analyzed using the Thrombin-Antithrombin (TAT) ELISA assay (TAT Ezygnost, Dade Berhing). Plasma was thawed at 37° C. for 15 minutes then used in the assay. Samples were run in duplicate.

Survival analysis of Activated Protein C and APC coagulation mutants at high and low doses. Mice were infused with either 10 μg or 2 μg of APC or an APC coagulation mutant as described above. Mice were given LPS via an i.p. injection and monitored for survival over a 7-day period.

Analysis of mouse plasma samples taken 24 hours after LPS infusion and treatment with recombinant APC. Mice (C57BI/6, EPCR^{δ/δ}, and PAR1^{-/-} were treated with 10 μg of recombinant APC or various APC mutants as the process was described above. Mice were given a (40 mg/kg) injection of LPS via an i.p. injection. Mice were then rested for 24 hours. 500 U of heparin was given and mice were bled through a vena cava draw. A small amount (−40 μl) of blood was used to measure blood cell numbers using the CBC machine. The remaining blood was spun down for 20 minutes at 2,000×g. Plasma was collected and flashed frozen in liquid nitrogen. Plasma was stored at −80° C. until use. Blood coagulation markers were measured by TAT, D-Dimer and Fibrinogen ELISA assays. Cytokines were measured using Bio-Rad's Bio-Plex system for mouse cytokines. A 23-plex system was used to measure 23 mouse cytokines as shown in Tables 1, 2, and 3. See FIGS. 10-12. Plasma was thawed at 37° C. for 15 minutes before use. Plasma was diluted 1/10 using Bio-Rad's serum dilutant for mouse plasma. Plasma was added to the filter plate with Bio-Plex beads. The plate was then read using the Illuminex 200 machine using the Bio-Plex manager program (Bio-Rad) for Windows.

Blood Collection and Histology. Blood collection from the vena cava was described previously in the cytokine analysis sections. Blood collected was measured using the Vet ABC counter from SCIL. Tissue was removed from the mouse and place in 10% neutral-buffered formalin solution. Tissue was then embedded flat in paraffin blocks and 4- to 5-μm longitudinal sections cut and stained with hematoxylin and eosin. Sections were viewed using Nikon Eclipse 600 for light microscopy and images were captured using the Spot Insight Camera and Spot advance software (Diagnostic Instruments, Sterling Heights, Mich.).

Flow Cytometry. Cells isolated from the spleen were analyzed in a LSRII flow cytometer (Becton-Dickson FACS Systems, Mountain View, Calif.). Briefly, samples of spleen were placed in sterile tubes containing 2 ml of RPMI 1640 (Life Technologies Inc., Grand Island, N.Y.). The tissue was processed with the homogenizer lab blender. The homogenized tissue was centrifuged and resuspended in 2 ml of 150 mM NH₄Cl, pH 7.0, to lyse red blood cells. After 2 min, the reaction was stopped by addition of 10 ml of PBS containing 1% (wt/vol) bovine serum albumin (BSA) and 0.1% (wt/vol) sodium azide (PBS-BSA-azide). Non-lysed cells were centrifuged and resuspended in PBS-BSA-azide. The resulting samples all had similar total numbers of cells. The cells were filtered through a 70-μm-pore-size nylon filter, and portions of the cells were stained with antibody-fluorochrome conjugates.

The following antibodies were purchased from BD Pharmingen, Inc. (San Diego, Calif.) and used for the flow cytometric analysis: allophycocyanin-conjugated anti-CD45 for all hematopoietic cells, fluorescein isothiocyanate-conjugated anti-CD4 and phycoerythrin-conjugated anti-CD8 for T cells, allophycocyanin-conjugated anti-Mac-1 for macrophages, phycoerythrin-conjugated anti-B220 for B cells, fluorescein isothiocyanate-conjugated anti-Gr1 for neutrophils, phycoerythrin-conjugated anti-NK1.1 and, fluorescein isothiocyanate-conjugated anti-CD3 for T cells. Cells were incubated on ice for 1 h in the presence of the appropriate antibodies, washed in PBS, and finally resuspended in ice-cold PBS containing 4% (wt/vol) paraformaldehyde, pH 7.4.

Prior to analysis, the cells were counted, and samples were adjusted to have the same cell number (2.5×10⁷ cells). All samples were initially gated on CD45 to identify hematopoietic cells. To analyze lymphocyte populations (T-cell subsets and B cells), CD45⁺ cells were gated by forward and side scatter, and then the total numbers of lymphocyte-sized cells that expressed the indicated surface marker were determined. Similarly, a gate based on forward and side scatter was set for the expected size of myeloid cells (monocytes/macrophages and granulocytes) prior to analysis of fluorescent markers. At least 30,000 gated events were acquired for each analysis.

TUNEL Assay. Tissue was removed from the mouse and place in 10% neutral-buffered formalin solution. Tissue was then embedded flat in paraffin blocks and 4-μm to 5-μm longitudinal sections cut and attached to slide. Slides were then deparaffinized by incubating slides overnight at 60° C. Slides were then incubated twice in xylene for 30 minutes each followed by successive washes in 100-95-70% ethanol for 1 minute each. Tissue was hydrolyzed by place in nanopure water twice for 5 minutes each. Tissue was then digested with 40 μg of proteinase K and then washed in 1×PBS. TUNEL assay was performed using In Situ cell death detection kit (Roche Applied Science, Indianapolis, Ind.) using the prescribed directions. Tissue sections were incubated for 60 minutes in a humidified chamber at 37° C. Tissue sections were then counterstained with Hoechst stain at (25 μg/7 mls) for 10 minutes. A drop of Vectashield (Vector Laboratories, Burlingame, Calif.) and a coverslip was added to each tissue section. Sections were visualized using the Zeiss Axioskop Fluorescent microscope (Zeiss, Thornwood, N.Y.) and images were captured using the Sensyc camera mounted to the microscope and analyzed using the Metamorph software (Molecular Devices, Sunnyvale, Calif.).

Caspase-3 Protease Assay. Caspase-3 protease activity in the liver was measured using the caspase-3 colorimetric assay kit (R&D systems, Minneapolis, Minn.) according the manufacture's instructions. Briefly, whole liver was weighed and homogenized in 1 ml of cell lysis buffer. Homogenates were then centrifuged for 1 minute at 10,000 g. The supernatant was incubated with Asp-Glu-Val-Asp-p-nitroanilide (pNA) and reaction buffer for 2 hours at 37° C. Levels of the chromophore pNA released by caspase-3 activity were quantified spectrophotometrically. The data was normalized by protein concentration per sample and are given as fold-increases in caspase-3 activity of experimental livers relative to non-LPS challenged control livers.

Dextran infusion studies. FITC-labeled high-molecular weight (MW) dextran (MW 200×10⁴, 100 mg/kg; Sigma) and rhodamine labeled low-MW dextran (MW 4×10⁴, 100 mg/kg; Sigma) were injected retroorbitally under 2.5% avertin anesthesia in mice 8 h after infusion of APC/LPS challenge, LPS challenge or vehicle (PBS). Fifteen minutes after dextran injection, the animals were killed and heart, lungs, kidney and liver were removed en bloc and immediately embedded in tissue freezing medium (Fisher Scientific), frozen in liquid nitrogen, and stored at −80° C. until further use. Tissue sections (10 μm) were cut and analyzed on a Nikon microscope equipped with a digital camera (Spot), an ultraviolet light source, and filters to visualize FITC and rhodamine fluorescence. All images were taken with the same camera settings and exposure times. Measurements were made using the Image J software (NIH).

Bacterial Sepsis model. Staphylococcus aureus (ATCC 29523) was grown from single colony isolates grown on tryptic soy agar (TSA). Isolates were grown in 5 mls of tryptic soy broth (TSB) overnight at 37° C. 1 ml of overnight culture was diluted into 20 mls of fresh TSB. The culture was grown for 3 hours to reach a log phase of growth. Culture was harvested by centrifugation (6000×rpm for 10 min) and wash once in 10 mls of PBS. After a second centrifugation, the pellet was resuspended in 1 ml of PBS. Cell numbers were determined by plate counts on TSA. 1×10⁸ bacteria were used for infection based on previous LD₅₀ experiments (data not shown). Mice were infused with either 10 μg or 2 μg of APC or various APC coagulation mutants as described above. For the additional infusion experiments, mice were anesthetized again and infused with APC or various mutants as previously described. Mice were then given bacteria via an i.p. injection at 1×10⁸ bacteria. Mice were then monitored for survival over a period of 7 days.

The references and patents cited herein are hereby incorporated by reference in their entirety.

We claim:

1. A method providing to a subject or to cells a dose of activated protein C or a variant of activated protein C wherein said dose is given as one or more bolus administrations.

2. The method of claim 1 wherein the variant of activated protein C is human 5A-ARC or murine 5A-APC.

3. The method of claim 1 wherein the variant of activated protein C is human KKK191-193AAA-APC or murine KKK192-194AAA-APC.

4. The method of claim 1 wherein the variant of activated protein C is human RR229/230AA-APC or murine RR230/231AA-APC.

5. The method of claim 1 wherein the activated protein C or a variant of activated protein C is given as a single bolus administration.

6. The method of claim 5 wherein the activated protein C or a variant of activated protein C is given as a single bolus administration over the course of about 20 minutes.

7. The method of claim 2 wherein the human 5A-APC or murine 5A-APC is given as a single bolus administration over the course of about 20 minutes.

8. The method of claim 3 wherein the human KKK191-193AAA-APC or murine KKK192-194AAA-APC is given as a single bolus administration over the course of about 20 minutes.

9. The method of claim 4 wherein the human RR229/230AA-APC or murine RR230/231AA-APC is given as a single bolus administration over the course of about 20 minutes.

10. The method of claim 1 wherein the activated protein C or a variant of activated protein C is given as two bolus administrations.

11. The method of claim 10 wherein the two bolus administrations consist of a first bolus administration and a second bolus administration.

12. The method of claim 11 wherein the second bolus administration is given to said subject at about 1 to about 10 hours after the first bolus administration is given to said patient.

13. The method of claim 12 wherein the dose of activated protein C or a variant of activated protein C in the first bolus administration and in the second bolus administration is about 0.067 to about 0.33 mg per kilogram of a subject's body mass.

14. The method of claim 13 wherein the variant of activated protein C is human 5A-ARC or murine 5A-APC.

15. The method of claim 1 wherein the dose of activated protein C or a variant of activated protein C is about 0.06 mg to about 0.4 mg per kilogram of a subject's body mass.

16. The method of claim 1 wherein the dose of activated protein C or a variant of activated protein C is about 0.067 to about 0.33 mg per kilogram of a subject's body mass.

17. The method of claim 1 wherein the variant of activated protein C has anticoagulant activity and cytoprotective activity, said activated protein C further having a protease domain comprising surface loops; wherein said activated protein C includes at least one mutation that differentially affects the activated protein C′s anticoagulant activity and cytoprotective activity, said at least one mutation being in at least one amino acid residue of a surface loop of said protease domain; wherein the said surface loop is selected from the group consisting of loop 37 and the calcium loop; and wherein said at least one mutation results in the anticoagulant activity, but not the cytoprotective activity, being reduced relative to a wild-type recombinant activated protein C.

18. The method of claim 2 wherein said subject has sepsis or severe sepsis.

19. The method of claim 2 wherein said subject has a bacterial infection.

20. A method providing to a subject or to cells a dose of a prodrug form of activated protein C or a prodrug form of a variant of activated protein C wherein said dose is given as one or more bolus administrations.

21. A method providing to a subject or to cells a dose of human 5A-APC or murine 5A-APC wherein said dose is given as one bolus administration.

22. The method of claim 21 wherein the dose of human 5A-APC or murine 5A-APC is about 0.067 to about 0.33 mg per kilogram of a subject's body mass.

23. The method of claim 21 wherein the human 5A-APC or murine 5A-APC is administered over the course of about 20 minutes.

24. A method providing to a subject a dose of human 5A-APC wherein said dose is given as one bolus administration over the course of about 20 minutes and wherein the dose is about 0.067 to about 0.33 mg per kilogram of the subject's body mass.
